(19) United States
(12) Patent Application Publication
(10) Pub. No.: US 2025/0187217 A1
(43) Pub. Date: Jun. 12, 2025

(54) DEVICE FOR DISCONNECTING AND SEALING A TUBE

(71) Applicant: Colder Products Company, Roseville, MN (US)

(72) Inventors: Kevin Porch, Apex, NC (US); Daniel A. Corazzo, Minneapolis,, MN (US); Neal W. Feldhahn, Oak Park Heights, MN (US); Jacob D. Fellows, Blaine, MN (US); Richard A. Kirchner, Apple Valley, MN (US); Isaac J. Milbrath, New Brighton, MN (US); Mark T. Nie, Elko, MN (US); Loi T. Truong, Savage, MN (US); Madeleine P. Turnberg, Minneapolis, MN (US); Grant A. Wilhelm, Maple Grove, MN (US); Randall S. Williams, Minneapolis, MN (US)

(21) Appl. No.: 18/972,474

(22) Filed: Dec. 6, 2024

Related U.S. Application Data
(60) Provisional application No. 63/606,855, filed on Dec. 6, 2023.

(51) Int. Cl. B26B 11/00; A61M 25/00

(52) U.S. Cl. CPC B26B 11/00; A61M 25/0015

(57) ABSTRACT

This document describes an improved disconnector device that is configured to attach to flexible tubing in a manner that compresses the flexible tubing and cuts the flexible tubing a compression point. The improved disconnector device can be in the form of a toolless aseptic disconnector configured to mate with an exterior of the flexible tubing.

### CROSS-REFERENCE TO RELATED APPLICATIONS

This application claims the benefit of U.S. Provisional Patent Application No. 63/606,855 filed on Dec. 6, 2023, the contents of which are fully incorporated herein by reference.

### TECHNICAL FIELD

This document relates to devices, systems and methods for cutting and sealing a tube, including for example, a sterile disconnector configured to engage and separate a tube at a selected location.

### BACKGROUND

Medical systems often utilize flexible tubing to transport biohazardous materials in a controlled and sterile manner. For instance, during hemodialysis, blood is drawn from a patient, passed through a dialyzer to filter waste, and returned to the body via flexible tubing, creating potential exposure to bloodborne pathogens. Similarly, intravenous (IV) systems use tubing to administer fluids, medications, or nutrients directly into a patient's bloodstream, where improper handling could lead to contamination. In laboratory settings, flexible tubing is employed in systems like bioreactors to transfer biological samples or infectious agents for research or diagnostics. Biopharmaceutical manufacturing systems can also use flexible tubing to carry chemical solutions that are potentially dangerous to humans. Additionally, surgical suction devices and drainage systems rely on tubing to remove blood, pus, or other infectious fluids from surgical sites. In each of these scenarios, strict protocols for handling biohazardous materials, sterilizing areas where biohazardous materials are present, and disposing biohazardous materials after use can be important for preventing contamination and ensuring safety.

Some fluid transfer systems for bioprocessing applications can transfer fluid through flexible tubing such as silicone tubing, plastic tubing such as polyvinyl chloride (PVC) tubing and thermoplastic elastomer (TPE) tubing, and synthetic rubber tubing such as ethylene propylene diene monomer (EPDM) tubing and fluoroelastomer (FKM) tubing. This fluid can, in some cases, include biohazardous material that is potentially dangerous to humans. In cases where flexible tubing carries biohazardous material, it can be beneficial to disconnect the tubing in a sterile manner so that fluid within the tubing does not leak from the tube following disconnection. For example, some sterile disconnection mechanisms require costly clamping equipment, such as a handheld tool that temporarily retains the tube during a cutting operation and is then detached from the tubing (for reuse of the tool at a later time).

### SUMMARY

Some embodiments described herein can include an improved disconnector device that is configured to attach to flexible tubing in a manner that compresses the flexible tubing and cuts the flexible tubing at a compression point. The improved disconnector device can be in the form of a toolless aseptic disconnector, which is configured to mate with an exterior of flexible tubing at a user-selected location, and then promptly clamp and cut the flexible tubing in manner that maintains sterility or otherwise avoids dripping or leakage from the cut ends of the tube.

The disconnector device further one or more cutting instruments that are positioned to cut the compressed tubing to separate the tubing into two parts. In some embodiments, the compression features and the cutting instrument can be integrated into a single part that does not require an external tool or reusable clamping hardware, and instead the single part includes structures that are movable and separable from one another so that a user can compress the tube at a user-selected location, safely cut the tube, and separate the tube at the cut ends in an aseptic manner. As described in further detail below, a user can place the disconnector device over the tube in a way that allows the user to compress the flexible tubing using only the disconnector device and without aid from additional compression devices separate from the disconnector device. Furthermore, a user can actuate the cutting instrument integrated into the disconnector device to cut the flexible tubing without the aid of any separate tools to perform the cutting. When the cutting is complete, the user can separate the flexible tubing by disconnecting the disconnector device into two parts. After the flexible tubing is separated, the device's disconnected parts maintain compression to prevent leakage from the flexible tubing.

One or more embodiments of a sterile disconnector for clamping, cutting, and separating flexible tubing are disclosed herein. In some cases, the sterile disconnector can be in the form of a toolless aseptic disconnector including a bottom structure and a top structure that are configured to attach to one another over flexible tubing in a way that compresses, or “clamps” the tube at a compression site. In some cases, the bottom structure can receive the flexible tubing within an open channel and the top structure can close over the bottom structure in a way that encapsulates the flexible tubing within the lumen and compresses the flexible tubing at a compression zone within the lumen. For instance, a user can place the bottom structure over the tubing and attach the top structure to the bottom structure at a hinge. Subsequently, the user can close the top structure over the bottom structure by rotating the top structure about the hinge to compress the flexible tubing. When the top structure is fully closed over the bottom structure, the top structure can attach to the bottom structure in a way that secures the housing portions together to prevent separation. In this attached position, the bottom structure and the top structure tightly compress the flexible tubing within the lumen. For example, the bottom structure and the top structure can compress the flexible tubing so that a lumen defined by the tubing is completely closed in a zone of compression, thus preventing liquid from crossing the zone of compression.

The cutting instrument of the sterile disconnector can, in some embodiments, cut the flexible tubing when the top structure is closed and securely attached over the bottom structure. The cutting instrument can, for example, occupy a retracted position before the tubing is cut where the blade is positioned within the housing proximate to the compressed tubing. To cut the tubing, the cutting instrument can advance into an aperture in the outer housing of the disconnector device in a way that causes the blade to cut completely through the flexible tubing in a compression zone where the flexible tubing is compressed. In some examples, the blade can cut through the flexible tubing between to or more compression zones so that at least one compression zone exists on either side of the cut. When the cut is complete, the cutting instrument occupies an actuated position where the cutting instrument extends fully into the outer housing of the disconnector device.

In some embodiments, the disconnector device ensures that sterility is maintained after cutting the flexible tubing in cases where the flexible tubing carries potentially biohazardous material such as human fluids, chemical formulations, medications, or any combination thereof. One way that the disconnector device provides sterility is by maintaining compression on both sides of the cut in the flexible tubing after the tube is separated. This ensures that liquid is contained within the lumen of the flexible tubing without escaping from the cut in the flexible tubing. To achieve this maintained compression, the disconnector device can separate at an interface perpendicular to the tubing, such that a first portion of the disconnector device's outer housing remains attached to the tubing on one end of the cut and a second portion of the disconnector device's outer housing remains attached to the tubing on the other end of the cut. The first portion of the disconnector device can maintain compression on the first end of the cut and the second portion of the disconnector device can maintain compression on the second end of the cut.

In one aspect, a device for cutting a tube can include an outer housing and a cutting instrument. The outer housing may include a bottom structure and a top structure attachable to the bottom structure at a first interface region. The bottom structure and the top structure can define a lumen sized to receive a tube. The cutting instrument may be slidably mounted within an aperture of the top structure to move from a retracted position to an actuated position to cut the tube. Optionally, a blade of the cutting instrument remains fully within the housing both when the cutting instrument is in the retracted position and when the cutting instrument is in the actuated position. The outer housing can be separable at a second interface region spaced apart from the first interface region when the bottom structure is attached to the top structure.

In another aspect, a device for cutting a tube can include an outer housing comprising a bottom structure comprising first pinching teeth and a top structure comprising second pinching teeth, the top structure attachable to the bottom structure at a first interface region. The bottom structure and the top structure can define a lumen sized to receive a tube and the first pinching teeth and the second pinching teeth compress the tube within the lumen. The device can also include includes a cutting instrument to cut the compressed tube a gap defined by the first pinching teeth and the second pinching teeth. The outer housing can be separable at a second interface region spaced apart from the first interface region, the second interface region extending through a gap defined by the first pinching teeth and the second pinching teeth.

In another aspect, a device for cutting a tube can include an outer housing comprising a bottom structure and a top structure movably attachable to the bottom structure at a first interface region, the bottom structure and the top structure defining a lumen sized to receive a tube. The device can also include a cutting instrument to cut the tube within the lumen, the outer housing being separable at a second interface region spaced apart from the first interface region based on a first side of the outer housing rotating relative to a second side of the outer housing so that connectors of the first side and the second side disengage, the first side of the outer housing remaining attached to the tube on a first end of the cut and the second side of the outer housing remaining attached to the tube on a second end of the cut.

In another aspect, a method, includes engaging a toolless aseptic disconnector with an exterior of flexible tubing at a user-selected location such that the toolless aseptic disconnector cuts the flexible tubing. The method can also include separating the toolless aseptic disconnector into a first part that is retained at a first cut end of the flexible tubing and a second part that is retained at a second cut end of the flexible tubing.

In another aspect, a method, can include mounting an aseptic disconnector to an exterior of flexible tubing. The method can also include twisting the aseptic disconnector into separate the aseptic disconnector into first and second pieces while the first and second pieces of the aseptic disconnector remain engaged with the flexible tubing.

Particular embodiments of the subject matter described in this document can be implemented to realize one or more of the following advantages. First, the disconnector device includes a clamping mechanism that can decrease a surface area of the flexible tubing that is clamped, thus decreasing an amount of force required to compress the flexible tubing. For example, the bottom structure of the disconnector device can include first pinching teeth and the second portion of the disconnector device can include second pinching teeth that face the first pinching teeth when the bottom structure is attached to the top structure. The first pinching teeth and the second pinching teeth can be placed adjacent to an interface where the disconnector device separates after the flexible tubing is cut. This ensures that the total clamping surface area is small and concentrated at or near a location where the cutting instrument cuts the flexible tubing. Because the clamping surface area is small, the user can manually close the housing mechanisms together without assistance from another tool separate from the disconnector device, as clamping force is concentrated on a small surface area rather than being spread over a larger surface area.

Second, to manually close the top structure over the bottom structure, the user can rotate the top structure relative to the bottom structure about a hinge until the housing portions are fully closed and secured together. The hinge mechanism can further reduce an amount of force necessary to clamp the tubing by providing leverage to translate the user's grip force into compression force applied to the tubing. For example, the part of the top and bottom structures that compress the tubing can be located between the hinge and a location where the user grips the top and bottom structures and squeezes the top and bottom structures to apply compression force. This means that when the user applies grip force, the hinge provides leverage to translate the grip force into compression. The hinge mechanism also allows for the clamping and cutting assembly to be closed at any desired position on the tubing and preferably provides tight tolerances for the hinging action which ensures proper alignment when actuated.

Third, when the bottom structure and the top structure close together to clamp the flexible tubing, the bottom structure and the top structure can secure the disconnector device to the flexible tubing in a way that prevents the disconnector device from separating from the tubing after the bottom structure and the top structure are closed together. When the tubing is compressed by the disconnector device's pinching teeth, the pinching teeth can provide a friction force that prevents the disconnector device from sliding laterally along the tubing (e.g., the disconnector device is fixed in one place along the tubing). This means that the user can select a location to later compress and cut the tubing. When the flexible tubing is cut, the disconnector device can separate into two parts including a right part and a left part, the right part maintaining compression force on a first end of the cut and the left part maintaining compression force on a second end of the cut. The compression forces prevent the first part from separating from the first end of the cut and prevent the second part from separating from the second end of the cut.

Fourth, a cutting instrument including a blade can be integrated into the clamping and cutting assembly such that, after the clamping and cutting assembly is closed onto the tubing, the user can activate the cutting assembly to cut the tubing manually without assistance from other tools separate from the disconnector device. In some embodiments, when the cutting instrument is in a retracted position and when the cutting instrument is in an actuated position, the blade can be fully enclosed within the outer housing of the disconnector device in a way that prevents the blade from being exposed to the user's skin and environment. That is, the outer housing can provide a barrier between the user and the blade that prevents the blade from injuring the user or others in the vicinity of the disconnector device before, during, and after cutting the flexible tubing.

Fifth, the disconnector device can include a quick-disconnect mechanism that allows the user to easily separate the disconnector device and tubing after the cut is performed. For example, the quick-disconnect mechanism can allow the user to twist the disconnector device so that the disconnector device can separate at an interface proximate to the cut in the flexible tubing. As described previously, this separation causes a left part of the disconnector device to separate from a right part of the disconnector device, with the left part maintaining compression force on one end of the cut and the right part maintaining compression force on the other end of the cut. This ensures that the tubing remains sterile, as potentially biohazardous liquid cannot escape through either end of the cut and contamination cannot enter the lumen of the tube through the cut.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIGS. 1A-1E show top, bottom, and side views of a disconnector device for clamping, cutting, and separating flexible tubing.

FIGS. 2A-2F show a process for using a disconnector device to disconnect flexible tubing.

FIGS. 3A-3B is a perspective view of a left portion of a disconnector device including twist disconnect receptacles.

FIG. 4 is a perspective view of a right portion of a disconnector device including twist disconnect posts.

FIGS. 5A-5I show examples of a disconnector device including mechanisms for retaining a cutting mechanism and flexible tubing in position.

FIGS. 6A-6D include perspective, side, and cutaway views of a disconnector device for compressing a tube.

FIGS. 7A-7D include perspective, side, and cutaway views of a disconnector device for cutting a compressed tube.

FIGS. 8A-8H show embodiments of twist disconnect receptacles and twist disconnect posts to allow a disconnector device to disengage to separate flexible tubing.

FIGS. 9A-13B show kickstand features for maintaining a top structure of a disconnector device in an open position relative to a bottom structure of the disconnector device.

FIGS. 14A-15C show an embodiment of a disconnector device including sliding tabs for preventing a cutting mechanism from actuating prematurely and preventing the disconnector device from disconnecting prematurely.

FIGS. 16A-16E show an embodiment of a disconnector device including a cutting mechanism with a rotating blade.

FIG. 17 shows an example where a bottom structure of a disconnector device is connected to tubing before the top structure is attached.

FIGS. 18A-18B show an anti-rotation mechanism for preventing a disconnector device from disconnecting prematurely.

FIGS. 19A-19B show an embodiment of a disconnector device including a blade that rotates about a central axis.

### DETAILED DESCRIPTION

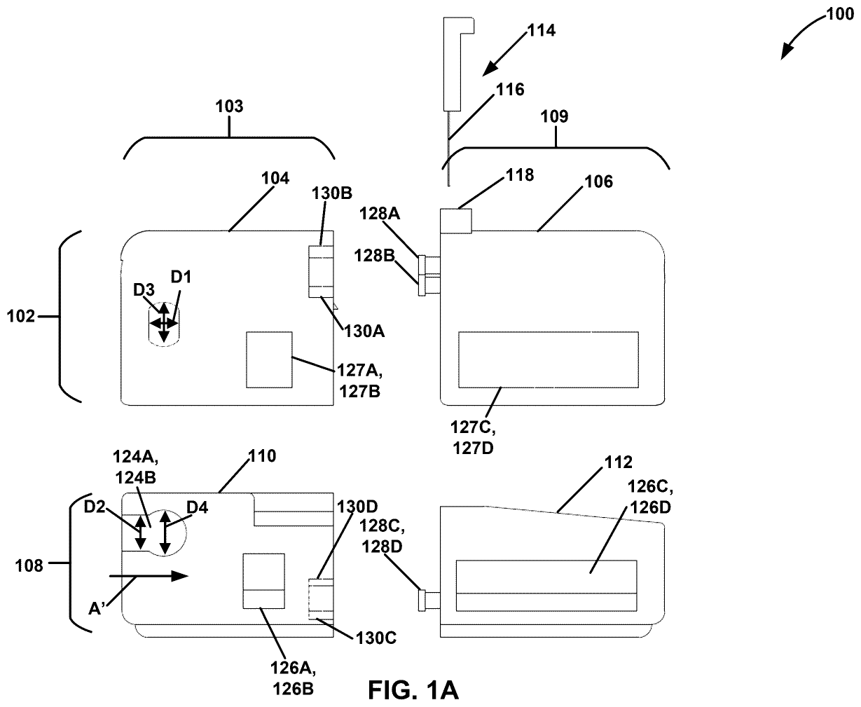

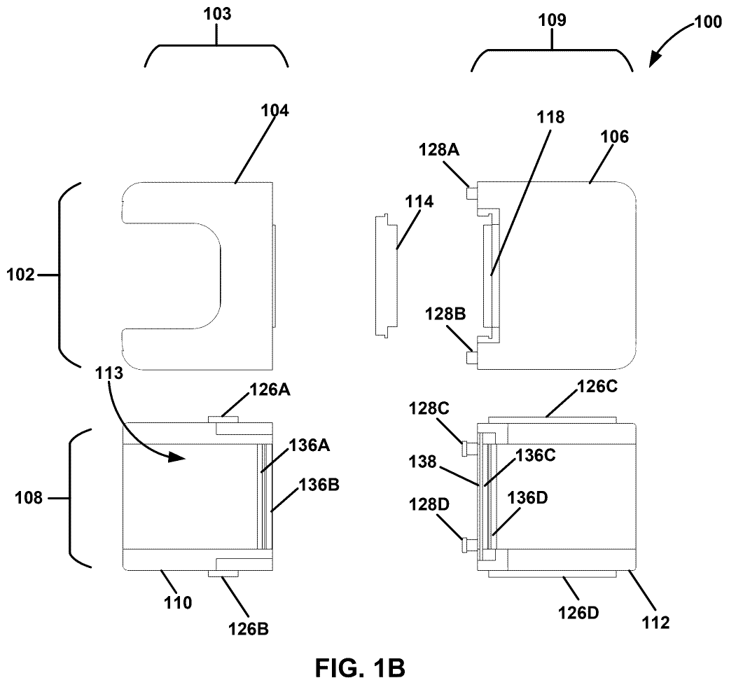

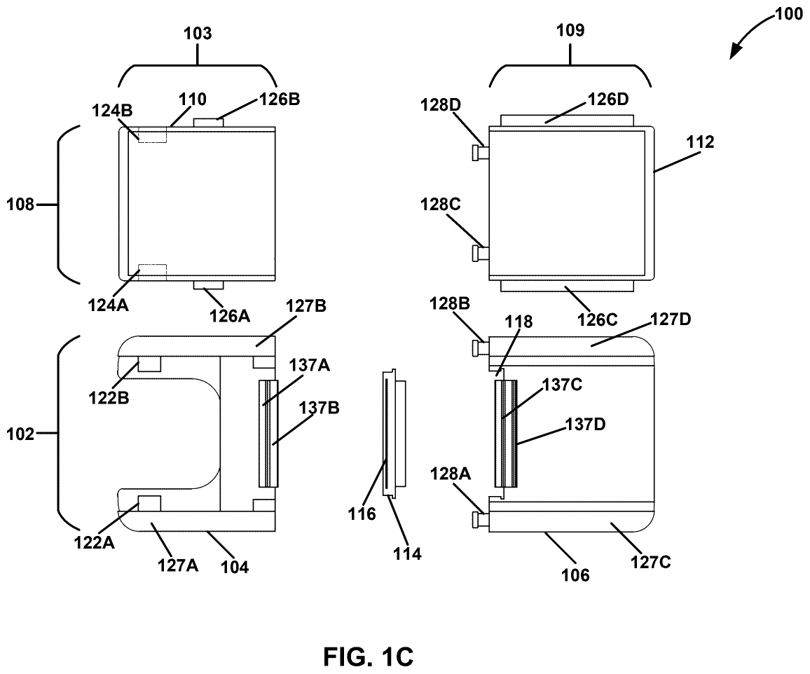

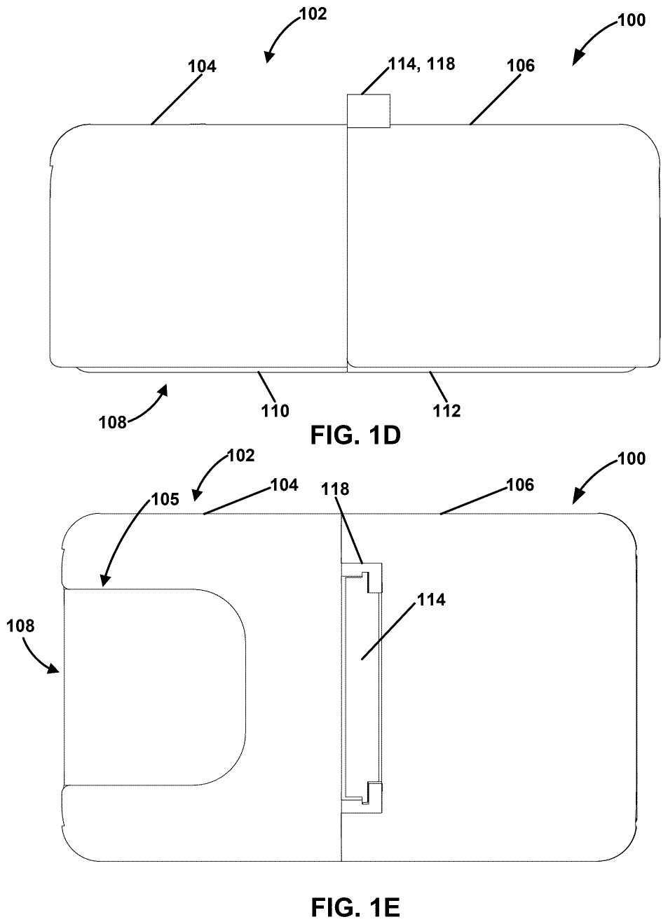

Referring now to FIGS. 1A-1C, some embodiments of a disconnector device 100 configured to clamp, cut, and separate flexible tubing can include a top structure 102 and a bottom structure 108. Disconnector device 100 can be attached at any location along flexible tubing that is sized to fit within a lumen defined by the top structure 102 and bottom structure 108 joined together so that top structure 102 and bottom structure 108 compress the tube within the lumen. In some embodiments, top structure 102 includes a first top component 104 and a second top component 106 that are removably attached to each other. Similarly, bottom structure 108 can, in some embodiments, include a first bottom component 110 and a second bottom component 112 that are removably attached to each other. In some examples, the first top component 104 and the second top component 106 of the top structure 102 are initially connected to each other and the first bottom component 110 and the second bottom component 112 of the bottom structure 108 are also initially connected to each other. In use, a user can connect the united top structure 102 and the united bottom structure 108 to compress flexible tubing in a way that completely closes a lumen of the flexible tubing at a compression zone.

Flexible tubing can include malleable materials such as silicone, plastic, rubber-like material, or any combination thereof. In medical and laboratory settings, flexible tubing often transports sterile fluids, gases, or media between devices or containers. To maintain sterility when the tubing is compressed, cut, and separated, disconnector device 100 can ensure that tubing is compressed to prevent fluid flow of biohazardous material. After flow is halted through compression, disconnector device 100 can sever the tubing, ensuring that both ends of the cut remain closed off to prevent contamination to the external environment and the fluid path. This process to compress, cut, and separate tubing is common in biopharmaceutical manufacturing and medical fluid administration systems, where maintaining a sterile pathway is important to human safety and product integrity.

In some embodiments, the bottom structure 108 defines an open channel 113 for receiving flexible tubing. That is, the open channel 113 can be sized to receive the flexible tubing so that the flexible tubing extends through the first top component 104 and the second top component 106 via the open channel 113. The top structure 102 can be sized to fit over the bottom structure 108 so that the top structure 102 and the bottom structure 108 compress the flexible tubing at a compression zone near a center of the disconnector device 100. For example, top structure 102 can define an open channel that is shaped to receive the bottom structure 108 so that top structure 102 can close over bottom structure 108. It is not required for the compression zone to be near the center of the disconnector device 100. In some cases, the compression zone can be displaced from the center of the disconnector device 100. For example, a user can fixedly attach the top structure 102 to the bottom structure 108 when the bottom structure 108 receives the flexible tubing in the open channel 113, thus securing the flexible tubing in the open channel 113 and compressing the flexible tubing at a compression zone.

The disconnector device 100 also includes a cutting instrument 114 including a blade 116 that passes through a cutting instrument recess 118 formed within the top structure 102 and bottom structure 108 so that blade 116 can cut the flexible tubing. In some examples, cutting instrument 114 can be slidably attached to the top structure 102 so that cutting instrument 114 can transition between a retracted position and an actuated position. In some embodiments, the cutting instrument 114 is attached to the top structure 102 so that blade 116 is located within a cross section of an outer casing of top structure 102 when cutting instrument 114 is in the retracted position, when cutting instrument 114 is in the actuated position, and when cutting instrument 114 is between the retracted position and the actuated position. This ensures that the outer casing of top structure 102 serves as a barrier between the blade and the user, thus decreasing a risk of injury. Even though FIGS. 1A-1C depict cutting instrument 114 as being separate from top structure 102, this is for illustration purposes and in some embodiments of disconnector device 100, blade 116 would be completely within the outer housing of top structure 102.

Top structure 102 can be secured to the bottom structure 108 through one or more mechanisms including a hinge which allows the top structure 102 to rotate relative to the bottom structure 108. For example, the top structure 102 includes hinge posts 122A-122B (collectively, “hinge posts 122”) located on an inner surface of the first top component 104. In some examples, hinge posts 122 include a first hinge post 122A located on a first side of first top component 104 and a second hinge post 122B located on a second side of first top component 104 (FIG. 1C). The bottom structure 108 defines hinge post receptacles 124A-124B (collectively, “hinge post receptacles 124”) in an outer surface of the first bottom component 110. In some examples, hinge post receptacles 124 include a first hinge post receptacle 124A located on a first side of first bottom component 110 and a second hinge post receptacle 124B located on a second side of first bottom component 110. The hinge posts 122 are configured to engage with the hinge post receptacles 124 so that the hinge posts 122 can rotate within the hinge post receptacles 124, thus causing top structure 102 to rotate about a hinge relative to bottom structure 108.

In some embodiments, an initial assembly of disconnector device 100 includes a unified top structure 102 where first top component 104 and second top component 106 are connected together and a unified bottom structure 108 where first bottom component 110 and second bottom component 112 are connected together. In some embodiments, top structure 102 and bottom structure 108 are initially separated. A user can connect the top structure 102 and the bottom structure 108 by sliding hinge posts 122 of top structure 102 into the hinge post receptacles 124 of bottom structure 108 while top structure 102 is perpendicular to bottom structure 108. For example, because the narrow dimension D1 of the hinge posts 122 is less than or equal to the narrow dimension D2 of hinge post receptacles 124, this means that hinge posts 122 can slide into hinge post receptacles 124 when top structure 102 and bottom structure 108 are oriented such that arrow A and arrow A′ face the same direction. When hinge posts 122 are inside hinge post receptacles 124, hinge posts 122 can rotate within hinge post receptacles 124 because the wide dimension D3 of hinge posts 122 is less than or equal to the wide dimension D4 of hinge post receptacles 124.

When the hinge posts 122 are inserted within hinge post receptacles 124, the top structure 102 can rotate relative to the bottom structure 108 about the hinge defined by hinge posts 122 and hinge post receptacles 124. Specifically, in some embodiments, the top structure 102 can rotate between an open position where the top structure 102 is substantially perpendicular to the bottom structure 108 with hinge posts 122 inside of hinge post receptacles 124 and a closed position where the top structure 102 is substantially parallel to bottom structure 108 with hinge posts 122 inside of hinge post receptacles 124. As the top structure 102 rotates from the open position to the closed position about the hinge, the top structure 102 and the bottom structure 108 can compress the flexible tubing within the open channel 113 defined by the bottom structure 108.

The hinge provides the user with leverage to manually compress the flexible tubing by rotating the top structure 102 relative to the bottom structure 108. For example, the user can grip a top surface of the second top component 106 and a bottom surface of the second bottom component 112 and press these two components together such that top structure 102 rotates inward relative to bottom structure 108 about the hinge. Pinching teeth one or more inside surfaces of top structure 102 and bottom structure 108 can compress the flexible tubing within the disconnector device 100, driven by the user's manual force in closing the top structure 102 and bottom structure 108 together. Disconnector device 100 allows a user to manually compress flexible tubing without assistance from tools separate from disconnector device 100. Thus, disconnector device 100 can be referred to as a “toolless” disconnector device.

In the closed position, top structure 102 is fully rotated inwards towards bottom structure 108 so that top structure 102 is parallel to bottom structure 108 with hinge posts 122 inside of hinge post receptacles 124. In the closed position, the hinge posts 122 can be oriented “upright” within the hinge post receptacles 124 so that the long direction of hinge posts 122 are perpendicular to a longitudinal axis of bottom structure 108. Because the long direction of hinge posts 122 are perpendicular to a longitudinal axis of bottom structure 108, hinge posts 122 are secured within the hinge post receptacles 124 because the narrow dimension D2 of hinge post receptacles 124 is too narrow to pass the long dimension D3 of hinge posts 122.

Furthermore, bottom structure 108 defines tabs 126A-126D (collectively, “tabs 126”) that are sized to engage with catches 127A-127D (collectively, “catches 127”) on an inside surface of top structure 102. For example, first bottom component 110 defines a tab 126A protruding from a first side of first bottom component 110 and a tab 126B protruding from a second side of first bottom component 110 (FIG. 1B). Second bottom component 112 defines a tab 126C protruding from a first side of second bottom component 112 and a tab 126B protruding from a second side of second bottom component 112 (FIG. 1B). Catches 127A-127D on the inside surface of top structure 102 can correspond to each of tabs 126A-126D on bottom structure 108 so that when top structure 102 closes fully onto bottom structure 108, tabs 126 engage with catches 127 to fixedly secure top structure 102 to bottom structure 108. For example, tab 126A can bond with catch 127A, tab 126B can bond with catch 127B, tab 126C can bond with catch 127C, and tab 126D can bond with catch 127D. In some cases, when top structure 102 fully closes onto bottom structure 108, pinching teeth 136, 137 can compress the tubing such that the flow path is closed off and the disconnector device 100 cannot be removed from the flexible tubing.

In some embodiments, the top structure 102 is permanently secured to bottom structure 108 so that when top structure 102 fully closes onto bottom structure 108 and tabs 126 engage with the catches 127, the first top component 104 and the first bottom component 110 cannot separate from each other and the second top component 106 and the second bottom component 112 cannot separate from each other. This is because when the tabs 126 and catches 127 bond, this can cause an irreversible connection due to the fact that lips of the tabs 126 face an inside surface of top structure 102 such that tabs 126 cannot be disengaged from catches 127. This disclosure is not limited to examples where a top structure and a bottom structure become irreversibly connected. In some examples, a top structure and a bottom structure can be separated after the top structure fully closes onto the bottom structure.

Disconnector device 100 also includes a quick-disconnect mechanism for disconnecting a left portion 103 of the disconnector device 100 and a right portion 109 of the disconnector device 100. As depicted in FIGS. 1A-1C, left portion 103 includes first top component 104 and first bottom component 110 and right portion 109 includes second top component 106 and second bottom component 112. The quick-disconnect mechanism can, in some examples, include twist disconnect posts 128A-128D (collectively, “twist disconnect posts 128”) and twist disconnect receptacles 130A-130D (collectively, “twist disconnect receptacles 130”). Twist disconnect posts 128 can engage with twist disconnect receptacles 130 so that the left portion 103 and the right portion 109 are connected to one another at an interface that is perpendicular to a longitudinal axis of disconnector device 100. For example, twist disconnect receptacle 130A can receive twist disconnect post 128A, twist disconnect receptacle 130B can receive twist disconnect post 128B, and so on. This can secure the left portion 103 to the right portion 109 so that the disconnector device 100 is whole before the side-by-side portions are disconnected.

Engagement between twist disconnect posts 128 and twist disconnect receptacles 130 can rely on an interlocking mechanism designed to align, secure, and disengage through rotational movement. In some embodiments, each of twist disconnect posts 128 can include a head and a shaft, with a head being wider than the shaft. Each of twist disconnect receptacles 130 can include a wide slot or opening greater than the width of the respective one of twist disconnect posts 128, allowing twist disconnect posts 128 to enter the wide slots of twist disconnect receptacles 130 when the left portion 103 and the right portion 109 are properly aligned. Once inserted, the objects can be twisted relative to each other, causing twist disconnect posts 128 to slide into narrow slots of twist disconnect receptacles 130. This engagement between twist disconnect posts 128 and twist disconnect receptacles 130 creates a firm mechanical hold by transferring the weight and force along the contact surfaces between the posts and receptacles.

In some cases, before top structure 102 closes onto bottom structure 108, first top component 104 can be connected to second top component 106 by twist disconnect posts 128A-128B and twist disconnect receptacles 130A-130B and first bottom component 110 can be connected to second bottom component 112 by twist disconnect receptacles 130A-130B. When top structure 102 closes onto bottom structure 108, first top component 110 can be fastened to first bottom component 104 to create left portion 103 and second top component 112 can be fastened to second bottom component 106 to create right portion 109. To separate right portion 103 and left portion 109, the portions can be twisted relative to one another, which disengages the locking features of twist disconnect posts 128 and twist disconnect receptacles 130, aligning the twist disconnect posts 128 with the wider entry slots of the twist disconnect receptacles 130. This rotational movement frees the posts to slide out of the receptacles, allowing the objects to separate.

The inner surfaces of first bottom component 110 and second bottom component 112 include pinching teeth 136A-136D (collectively, “pinching teeth 136”), as depicted in FIG. 1B. The pinching teeth 136 can extend inwards from the inner surface of bottom structure 108 so that pinching teeth 136 protrude within the open channel 113 of the bottom structure 108. Pinching teeth 136A and 136B extend along an inner surface of first bottom component 110 perpendicular to a longitudinal axis of disconnector device 100. The pinching teeth 136 can extend inwards from the inner surface of bottom structure 108 so that pinching teeth 136 protrude within the open channel 113 of the bottom structure 108. Similarly, pinching teeth 136C and 136D extend along an inner surface of second bottom component 112 perpendicular to a longitudinal axis of disconnector device 100. In some embodiments, pinching teeth 136 are located proximate to an interface between first bottom component 110 and second bottom component 112 so that pinching teeth 136 compress the flexible tubing at a location where blade 116 cuts the flexible tubing.

The inner surfaces of first top component 104 and second top component 106 include pinching teeth 137A-137D (collectively, “pinching teeth 137”), as depicted in FIG. 1C. The pinching teeth 137 can extend inwards from the inner surface of top structure 102 so that pinching teeth 137 protrude within top structure 102. Pinching teeth 137A and 137B extend along an inner surface of first top component 104 perpendicular to a longitudinal axis of disconnector device 100. Similarly, pinching teeth 137C and 137D extend along an inner surface of second top component 106 perpendicular to a longitudinal axis of disconnector device 100. In some embodiments, pinching teeth 137 are located proximate to an interface between first bottom component 110 and second bottom component 112 so that pinching teeth 137 compress the flexible tubing at a location where blade 116 cuts the flexible tubing.

In some embodiments, when top structure 102 is closed onto bottom structure 108, pinching teeth 136 and pinching teeth 137 are configured to compress the flexible tubing as top structure 102 rotates relative to bottom structure 108 from the open position to the closed position. When top structure 102 reaches the closed position where top structure 102 is parallel to bottom structure 108 and the tabs 126 engage with the catches 127, pinching teeth 136 and pinching teeth 137 can fully compress the flexible tubing so that liquid cannot pass through the compression zone. Because pinching teeth 136 and pinching teeth 137 are concentrated at a center of disconnector device 100 and are relatively short as compared with a total length of disconnector device 100, a user can manually compress flexible tubing using disconnector device 100 without assistance from a tool separate from disconnector device 100. This is because pinching teeth 136 and pinching teeth 137 concentrate the user's manual force on a small surface area of the flexible tubing during compression so the force is not spread out over a larger area which would require a greater amount of total force for compression.

A valley can be defined between each pair of pinching teeth on disconnector device 100. In some embodiments, a valley is defined between pinching teeth 136A and 136B, a valley is defined between pinching teeth 136C and 136D, a valley is defined between pinching teeth 137A and 137B, and a valley is defined between pinching teeth 137C and 137D. These valleys can increase an amount of frictional force that pinching teeth 136 and 137 apply to the flexible tubing when compressed and therefore decrease a likelihood that disconnector device 100 will become dislodged from the flexible tubing. The valleys also can further reduce a surface area of the pinching teeth 136 and 137 that contact the flexible tubing during compression, thus decreasing an amount of force necessary to compress the tube. For example, because the user's manual force in closing the top structure 102 onto the bottom structure 108 is focused on the parts of pinching teeth 136 and 137 that are in contact with the flexible tubing, the valleys can help to decrease a surface area in contact with the flexible tubing and therefore decrease the amount of force needed to fully compress the tube. In some examples, pinching teeth 136 and 137 can have similar profiles (e.g., both flat or both serrated). In some examples, pinching teeth 136 and 137 can have different profiles (e.g., one can be serrated and one can be flat). In these examples, serrated pinching teeth can provide mechanical holding power and flat pinching teeth can provide compression.

In some examples, when the top structure 102 is in the closed position so that top structure 102 and bottom structure 108 are parallel and tabs 126 are engaged with catches 127, there is a narrow gap between pinching teeth 136 and pinching teeth 137. This narrow gap, in some examples, defines a width that is significantly smaller than a diameter of the flexible tubing when it is not compressed. In some examples, a width of the narrow gap is less than or equal to twice the width of the flexible tubing wall. This means that when the flexible tubing is fully compressed so that opposing walls of the tubing are pressed together, the tube can fit within the narrow gap. In some examples, when the top structure 102 is in the closed position over the bottom structure 108, pinching teeth 136A and 136B are opposite pinching teeth 137A and 137B and pinching teeth 136C and 136D are opposite pinching teeth 137C and 137D.

The cutting instrument 114 can be slidably attached to the second top component 106 of the top structure 102. When the second top component 106 is attached to the first top component 104, cutting instrument 114 sized to slide within cutting instrument recess 118 formed in the surface of the top structure 102. For example, cutting instrument 114 can be sized to slide from a retracted position where a handle portion of the cutting instrument 114 extends outward from the top structure 102 to an actuated position where the handle portion is inserted into the cutting instrument recess 118. This transition from the retracted position to the actuated position can occur when the user actuates the cutting instrument 114 to cut the flexible tubing.

In some embodiments, when the top structure 102 is fully closed onto the bottom structure 108 to compress the flexible tubing between pinching teeth 136 and pinching teeth 137 and the cutting instrument 114 is in the retracted position, pinching teeth 136 is within the outer housing of disconnector device 100 proximate to the flexible tubing. To cut the flexible tubing the user can apply pressure to the handle portion of the cutting instrument 114 that extends outwards from the top structure 102 to press the cutting instrument 114 into the outer housing of the disconnector device 100. This can cause the blade 116 to cut through the flexible tubing as cutting instrument 114 advances. In some embodiments, the blade 116 cuts the flexible tubing in a location where pinching teeth 136 and pinching teeth 137 compress the flexible tubing. The user can, in some examples, advance the cutting instrument 114 to a position where the blade 116 rests within a protective blade recess 138 that is on an opposite side of the flexible tubing in a cross section of disconnector device 100 from a handle portion of the cutting instrument 114.

Cutting instrument 114 can cut the flexible tubing at a location near a center of the pinching teeth 136 and pinching teeth 137 along a longitudinal axis of disconnector device 100. For example, as depicted in FIG. 1B, protective blade recess 138 is located between pinching teeth 136A, 136B and pinching teeth 136C, 136D. That is, protective blade recess 138 is to the right of pinching tooth 136B and to the left of pinching tooth 136C in the view of FIG. 1B. This means that blade 116 cuts through the compressed flexible tubing at a location between pinching tooth 136B and pinching tooth 136C and passes into the protective blade recess 138. After the cut is made, pinching teeth 136A, 136B and pinching teeth 137A, 137B maintain compression force on a left side of the cut and pinching teeth 136C, 136D and pinching teeth 137C, 137D maintain compression force on a right side of the cut. This prevents liquid from flowing out of the cut made by the blade 116 passing through the flexible tubing and into the protective blade recess 138.

After the cutting instrument 114 transitions from the retracted state to the actuated state to cut through the compressed flexible tubing, the user can disconnect the left portion 103 from the right portion 109 using the quick-disconnect mechanism of disconnector device 100. For example, because the flexible tubing is completely cut through within the outer housing of disconnector device 100, the user can rotate the right portion 109 relative to the left portion 103 without the flexible tubing itself stopping rotation. Disconnector device 100 can also include an interlock feature prevents rotation until blade 116 is fully depressed. When right portion 109 of disconnector device 100 rotates relative to left portion 103, the twist disconnect posts 128 can disengage from the twist disconnect receptacles 130 so that left portion 103 and right portion 109 can be separated.

When left portion 103 and right portion 109 are separated, first top component 104 and first bottom component 110 can remain connected to each other and second top component 106 and second bottom component 112 can remain connected to each other. This ensures that pinching teeth 136A, 136B and pinching teeth 137A, 137B maintain compression force on a left side of the cut and pinching teeth 136C, 136D and pinching teeth 137C, 137D maintain compression force on a right side of the cut following separation. After the left portion 103 and right portion 109 are separated, cutting instrument 114 can remain in the actuated position where a sharp portion of blade 116 is housed within the protective blade recess 138. In some cases, left portion 103 includes one or more features that prevent axial movement of first top component 104 and first bottom component 110 relative to each other after separation. In some cases, right portion 109 includes one or more features that prevent axial movement of second top component 106 and second bottom component 112 relative to each other after separation.

Referring now to FIGS. 1D-1E, disconnector device 100 represents a single integrated piece when the top structure 102 is fully closed over the bottom structure 108. FIG. 1D depicts a side view of disconnector device 100 where the top structure fully extends over the bottom structure 108 except for the bottom surfaces of first bottom component 110 and second bottom component 112 which are exposed. In this way, the outer surfaces of top structure 102 and the outer bottom surface of bottom structure 108 together can represent an outer housing of disconnector device 100 when top structure 102 is fully closed over the bottom structure 108. FIG. 1E depicts a top view of disconnector device 100 when top structure 102 is fully closed over the bottom structure 108. As depicted in FIG. 1E, the first top component 104 includes a window 105 through which the bottom structure 108 is visible. This window 105 allows the user to see the flexible tubing within disconnector device 100. FIGS. 1D-1E depict cutting instrument 114 in the actuated position where the handle portion of cutting instrument 114 is fully extended into disconnector device 100. This is the position that cutting instrument 114 can occupy after cutting the flexible tubing. In some examples, there can be a visual indicator that cutting instrument 114 is in the actuated position. For example, disconnector device 100 can include a window through which a position of cutting instrument 114 is visible.

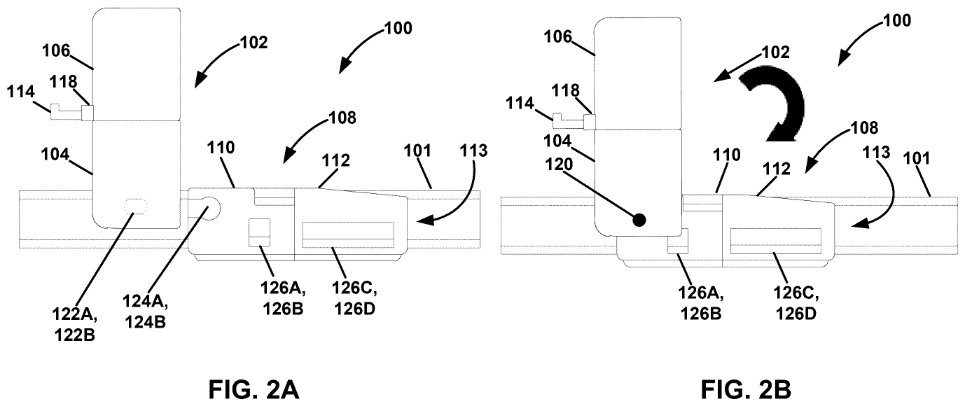

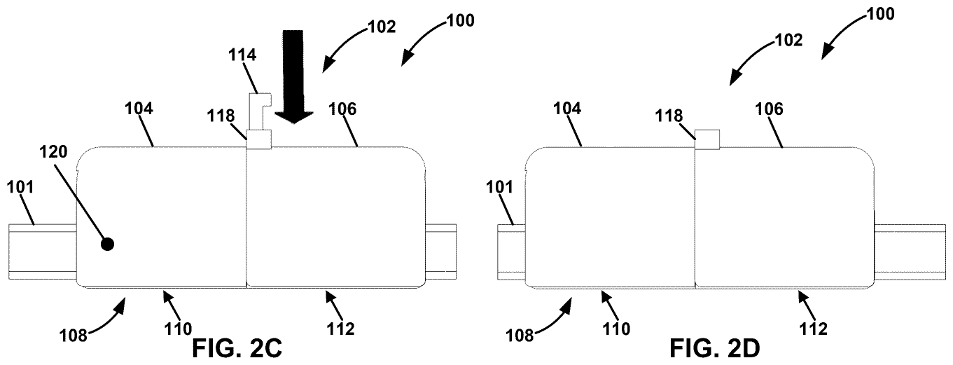

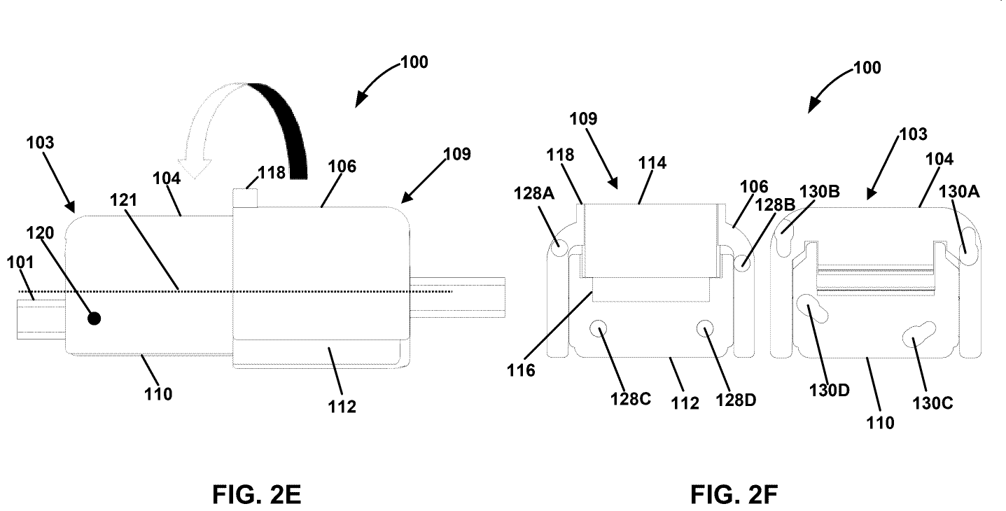

Referring now to FIGS. 2A-2F, disconnector device 100 can occupy various configurations as disconnector device 100 secures to flexible tubing 101, cuts the flexible tubing 101, and separates the flexible tubing 101. For example, FIG. 2A depicts disconnector device 100 when top structure 102 and bottom structure 108 are separate pieces, with first top component 104 and second top component 106 being connected to define top structure 102 and first bottom component 110 and second bottom component 112 being connected to define bottom structure 108. The bottom structure 108 can be sized to receive the flexible tubing 101 in an open channel 113. As depicted in FIG. 2A, top structure 102 defines hinge posts 122 and bottom structure 108 defines hinge post receptacles 124 that are sized to receive the hinge posts 122 so that top structure 102 can rotate relative to bottom structure 108 about a hinge.

Specifically, in some embodiments, top structure 102 can be positioned perpendicular to bottom structure 108 and the flexible tubing 101 as depicted in FIG. 2A. This orients the hinge posts 122 so that the narrow dimension of hinge posts 122 are aligned with the openings of hinge post receptacles 124. In this orientation, the hinge posts 122 of top structure 102 can slide into the hinge post receptacles 124 of bottom structure 108. When the top structure 102 slides onto the bottom structure 108, top structure 102 and bottom structure 108 can form an “L” shape with flexible tubing 101 passing through the open channel 113 of bottom structure 108 as depicted in FIG. 2B. When the top structure 102 slides onto the bottom structure 108 to occupy the position depicted in FIG. 2B, this allows the hinge posts 122 to rotate within the circular section of the hinge post receptacles 124 so that top structure 102 can close onto the bottom structure 108 with hinge posts 122 remaining within hinge post receptacles 124 and the flexible tubing 101 remaining within the open channel 113 of bottom structure 108.

The cutting instrument 114 can occupy the retracted position until the top structure 102 is fully closed onto the bottom structure 108 so the flexible tubing 101 is compressed before cutting. This ensures that the blade 116 does not cut the flexible tubing 101 or become exposed to the user before the tubing is adequately compressed, thus preserving a sterile environment by preventing a situation where biohazardous material leaks out of an inadequately compressed tube or where contamination enters the tube. For example, when top structure 102 transitions from the open position depicted in FIG. 2B to the closed position depicted in FIG. 2C, the cutting instrument 114 remains in the retracted position while the pinching teeth 136, 137 compress the flexible tubing 101 so that blade 116 does not cut the flexible tubing 101 during compression. FIGS. 2A-2C each depict the cutting instrument 114 in the retracted position, with a portion of the handle of cutting instrument 114 protruding outward from the outer surface of disconnector device 100. In some examples, top structure 102 rotates relative to bottom structure 108 about a hinge axis 120.

In some embodiments, top structure 102 transitions from the open position depicted in FIG. 2B to the closed position depicted in FIG. 2C by rotating about the hinge created by hinge posts 122 being inserted within hinge post receptacles 124. A user can manually perform this transition by applying pressure to a surface of second top component 106 to cause second structure 102 to rotate clockwise relative to bottom structure 108 about the hinge. When top structure 102 reaches the closed position depicted in FIG. 2C, tabs 126 can engage with catches 127 to cause top structure 102 and bottom structure 108 to fasten together in the closed position. In this closed position, the pinching teeth 136, 137 can fully compress the flexible tubing 101.

To cut the flexible tubing 101, cutting instrument 114 can advance into the outer housing of disconnector device 100 from the retracted position depicted in FIG. 2C to the actuated position depicted in FIG. 2D. To advance from the retracted position to the actuated position, the handle portion of the cutting instrument 114 that is initially outside of the outer housing in the retracted position can advance into the cutting instrument recess 118 until the handle is nearly or completely within the outer housing. This causes the blade 116 attached to the handle portion of cutting instrument 114 to advance through disconnector device 100, cutting through the flexible tubing 101 at a location where flexible tubing 101 is compressed by pinching teeth 136, 137. After the cut is performed, pinching teeth 136, 137 can maintain compression on both sides of the cut so that biohazardous liquid does not leak out of the cut and contamination cannot enter the tubing. In use, a user can advance cutting instrument 114 from the retracted position depicted in FIG. 2C to the actuated position depicted in FIG. 2D by applying pressure to the handle of cutting instrument 114 in the downward direction of FIG. 2C′s arrow. This downward pressure causes the blade 116 to advance and cut through the cutting instrument 114.

After cutting instrument 114 cuts the flexible tubing 101, a user can rotate the right portion 109 of disconnector device 100 relative to the left portion 103 about an axis parallel to the flexible tubing 101. This rotation is depicted in FIG. 2E, with first top component 104 and first bottom component 110 aligned in a first rotational position and the second top component 106 and the second bottom component 112 aligned in a second rotational position that is offset from the first rotational position. The rotation can occur because twist disconnect posts 128 disengage from twist disconnect receptacles 130 when right portion 109 rotates relative to left portion 103. This allows right portion 109 to separate from left portion 103 as depicted in FIG. 2F. In some examples, left portion 103 can maintain a compression force on one side of the cut and right portion 109 can maintain a compression force on another side of the cut following separation of right portion 109 and left portion 103. In some examples, right portion 109 rotates relative to left portion 103 about a rotation axis 121. The rotation axis 121, in some examples, forms an angle with the hinge axis 120. For example, rotation axis 121 can be perpendicular to hinge axis 120.

As depicted in FIG. 2F, the twist disconnect receptacles 130 can each include a small circular portion and a large circular portion. When disconnector device 100 is in the position depicted in FIG. 2D where the left portion 103 is rotationally aligned with the right portion 109, the twist disconnect posts 128 can be received within the respective small circular portions of twist disconnect receptacles 130. These small circular portions of twist disconnect receptacles 130 can include a lip or cam that engages with the respective twist disconnect post of twist disconnect posts 128 to prevent left portion 103 for separating form right portion 109. When right portion 109 rotates relative to left portion 103, this causes twist disconnect posts 128 to dislodge from the small circular portions of twist disconnect receptacles 130 to enter the large circular portions of twist disconnect receptacles 130. In some examples, a diameter of the large circular portions of twist disconnect receptacles 130 is larger than a diameter of the twist disconnect posts 128, meaning that twist disconnect posts 128 can move laterally out of twist disconnect receptacles 130 along an axis parallel to the flexible tubing 101. Twist disconnect receptacles 130 are not limited to including circular portions. The portions can be shaped differently if heads on the twist connection posts 128 are not circular in shape.

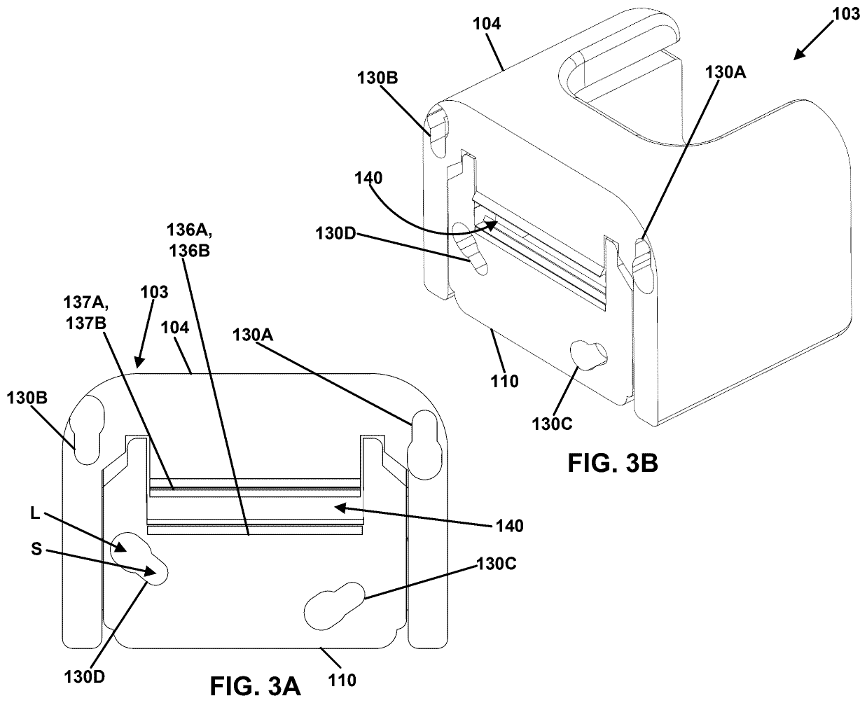

FIGS. 3A-3B provide additional views of the left portion 103 of disconnector device 100 when first top component 104 is attached to first bottom component 110. As depicted in FIGS. FIGS. 3A-3B, each of twist disconnect receptacles 130 includes a large circular portion (labeled “L” in the example of twist disconnect receptacle 130D) and a small circular portion (labeled “S” in the example of twist disconnect receptacle 130D). The small circular portions can tightly secure the twist disconnect posts 128 within twist disconnect receptacles 130, whereas the large circular portions are wide enough to allow the twist disconnect posts 128 to slide out of the twist disconnect receptacles 130. On left portion 103, the twist disconnect receptacles 130 are positioned so that twist disconnect posts 128 of right portion 109 can rotate in unison out of the small circular portions of twist disconnect receptacles 130 and into the large circular portions of twist disconnect receptacles 130. There exists a gap 140 between pinching teeth 136A, 136B of first bottom component 110 and pinching teeth 137A, 137B of first top component 104. The flexible tubing 101 can be fully compressed in this gap so that liquid does not leak out of a cut in the flexible tubing 101 and so that contamination does not enter the flexible tubing 101.

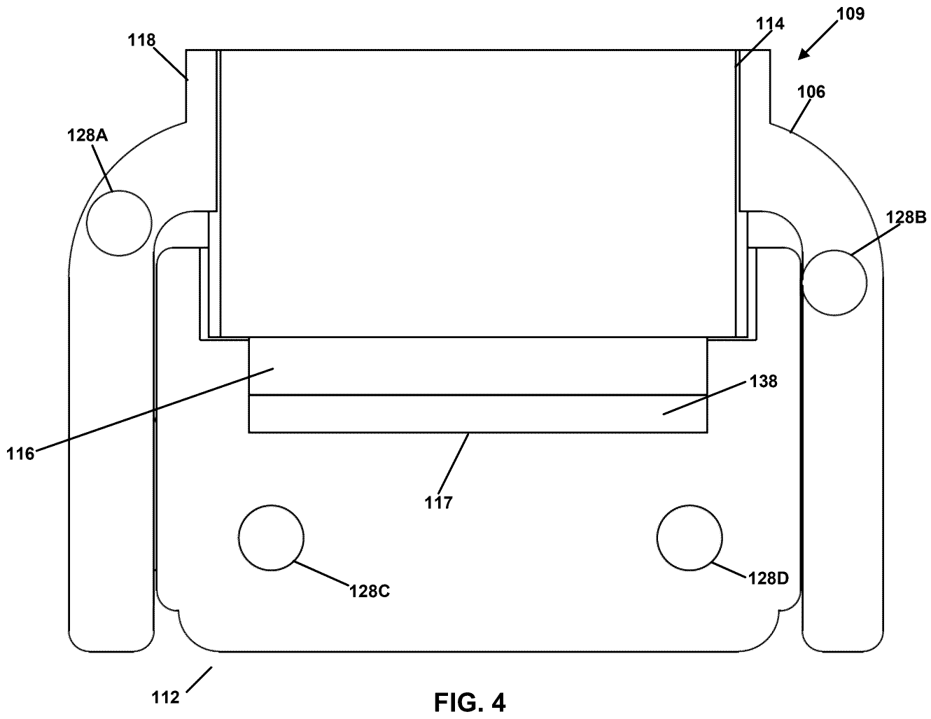

FIG. 4 provides an additional view of the right portion 109 of disconnector device 100 when second top component 106 is attached to second bottom component 112. As depicted in FIG. 4, each of twist disconnect posts 128 includes a circular cross-section. In some cases, the circular cross-section of each twist disconnect post 128 can fit snugly within the small circular portion of the respective twist disconnect receptacle 130. This means that when twist disconnect posts 128 are received within the small circular portions of twist disconnect receptacles 130, right portion 109 and left portion 103 resist separation even if a pulling force is applied to either or both ends of disconnector device 100. However, when right portion 109 is rotated relative to left portion 103, twist disconnect posts 128 can move simultaneously out of the small circular portions of the twist disconnect receptacles 130 and into the large circular portions of the twist disconnect receptacles 130. In some embodiments, the larger diameter of the large circular portions does not snugly fit to the diameter of twist disconnect posts 128, meaning that right portion 109 can easily separate from left portion 103. As depicted in FIG. 4, blade 116 of the cutting instrument 114 can fit within protective blade recess 138 to protect the user from injury.

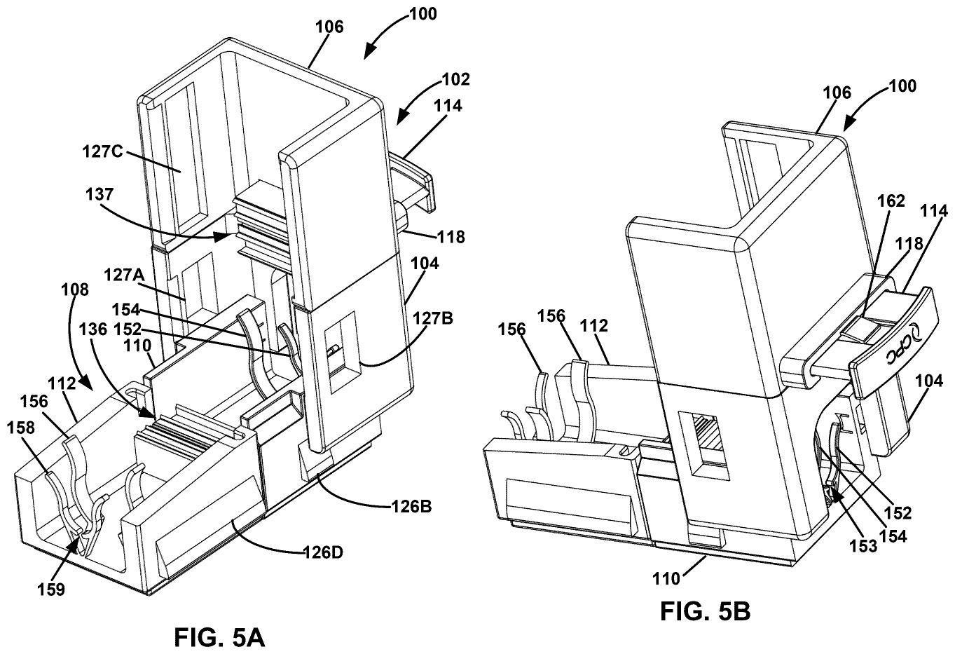

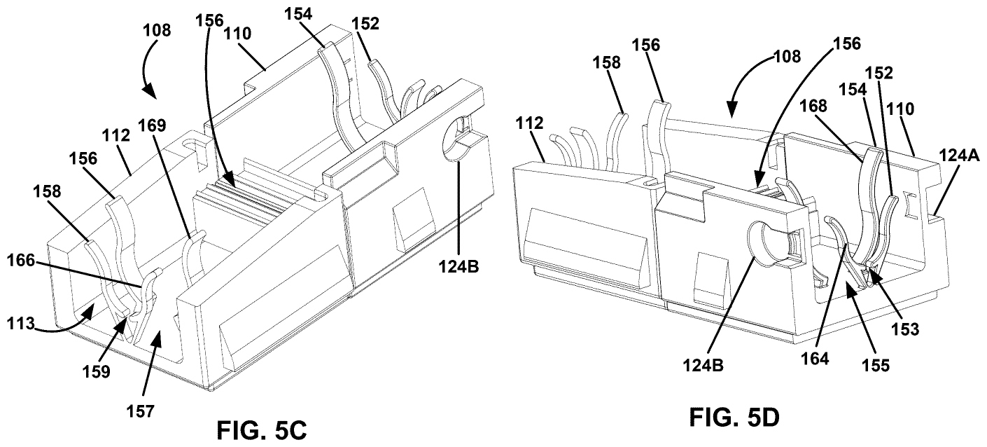

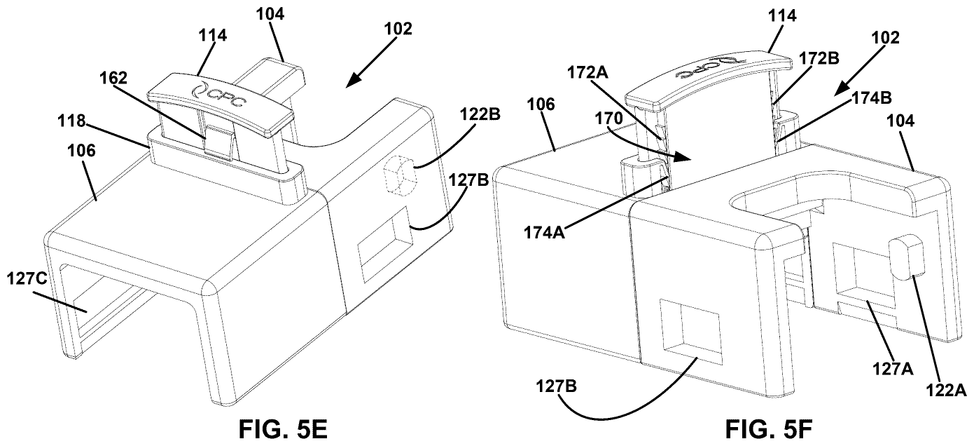

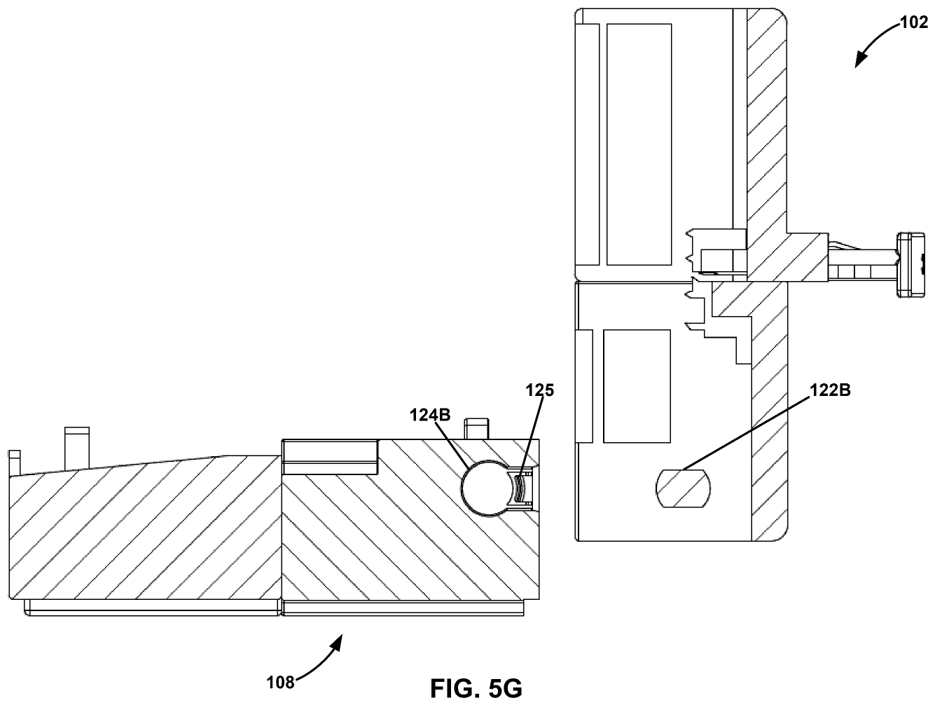

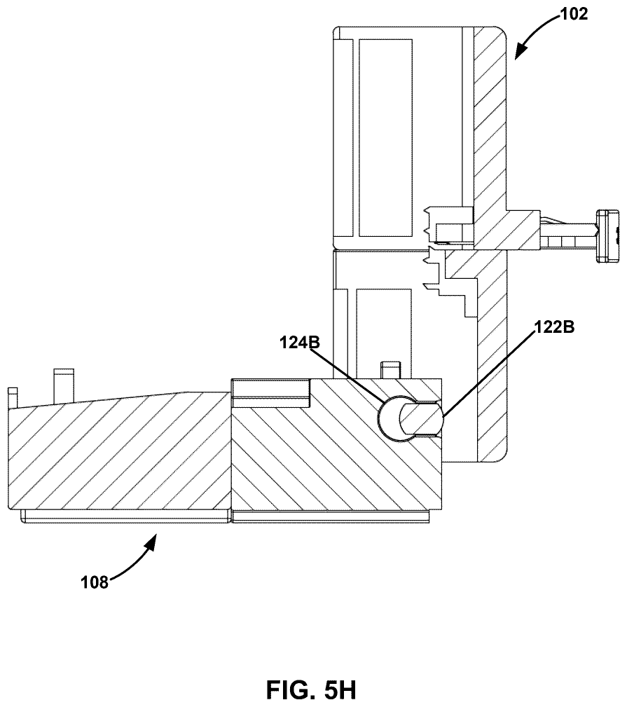

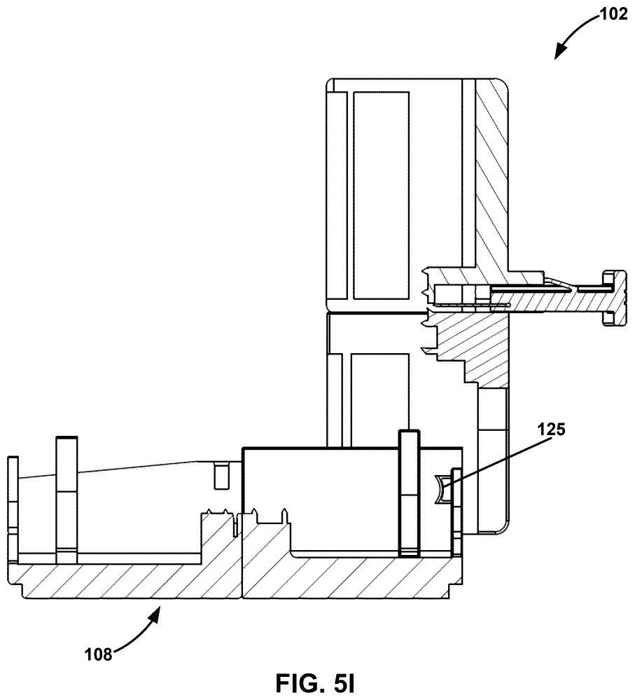

Referring now to FIGS. 5A-5B, the disconnector device 100 can include one or more features securing the tube, within the disconnector device 100, securing the blade in a raised or lowered position, securing the top structure 102 of the disconnector device 100 to the bottom structure 108 of the disconnector device 100, or any combination thereof. For example, disconnector device 100 includes a first small cradle 152, a first large cradle 154, a second large cradle 156, and a second small cradle 158. The first small cradle 152 and the second small cradle 158 are sized to receive a first size of flexible tubing and the first large cradle 154 and the second large cradle 156 are sized for a second size of flexible tubing larger than the first size of flexible tubing.

Cradles 152-158 are configured to secure flexible tubing at a position within the disconnector device 100, particularly before top structure 102 closes onto bottom structure 108 so that pinching teeth 136, 137 compress the tubing. For example, the first small cradle 152 and the second small cradle 158 are configured to receive a smaller-sized flexible tubing such that the flexible tubing is supported by first small cradle 152 and second small cradle 158 while passing through first large cradle 154 and the second large cradle 156. That is, when the diameter of first large cradle 154 and the second large cradle 156 is larger than the diameter of the smaller-sized flexible tubing, the flexible tubing can be supported by first small cradle 152 and second small cradle 158 without interference from first large cradle 154 and the second large cradle 156. By supporting the smaller-sized flexible tubing within the bottom structure 108 before top structure 102 closes onto bottom structure 108, the first small cradle 152 and second small cradle 158 can advantageously secure the bottom structure 108 to the flexible tubing so the user can position to close the disconnector device 100, thus decreasing a likelihood that bottom structure 108 falls off the tubing before the disconnector device 100 is closed.

In some embodiments, first large cradle 154 and second large cradle 156 are configured to receive a larger-sized flexible tubing to support the flexible tubing within bottom structure 108 before the top structure 102 closes onto the bottom structure 108. Even though a resting diameter of the first small cradle 152 and second small cradle 158 can be smaller than a diameter of the larger-sized flexible tubing, the first small cradle 152 and second small cradle 158 can expand to accommodate the larger-sized tubing while the first large cradle 154 and second large cradle 156 also grip the larger-sized flexible tubing. For example, first small cradle 152 defines a gap 153 that allows arm portions of the first small cradle 152 to separate from each other to accommodate larger tubing. Second small cradle 158 defines a gap 159 that allows arm portions of the second small cradle 158 to separate from each other to accommodate larger tubing. This means that when bottom structure 108 receives the larger-sized tubing, the arms of the first small cradle 152 and second small cradle 158 can bend outwards to accommodate the tubing, whereas the first large cradle 154 and second large cradle 156 can accommodate the larger-sized tubing without similar significant bending.

As depicted in FIG. 5B, the cutting instrument 114 can include a retention mechanism 162 that secures the cutting instrument 114 in the retracted position before the cutting instrument 114 is actuated to cut the flexible tubing. Maintaining the cutting instrument 114 in the retracted position before use can be advantageous for safety reasons, so that the blade 116 does not become prematurely exposed during shipping or at any other time before the user intends to actuate the blade. In the retracted position, the blade 116 can be concealed within top structure 102 as depicted in FIG. 5A, thus protecting the user from the sharp end of the blade 116. This means that it is beneficial to maintain the cutting instrument 114 in the retracted position.

Retention mechanism 162 can protrude onto a lip of cutting instrument recess 118, as depicted in FIG. 2B. Because retention mechanism 162 protrudes onto the lip of cutting instrument recess 118, this prevents the cutting instrument 114 from advancing into the top structure 102 before the user is ready to actuate the cutting instrument 114. The retention mechanism 162 therefore can retain cutting instrument 114 in the retracted position depicted in FIG. 5B, even when a force is applied to cutting instrument 114 downwards towards the top structure 102. The blade 116 remains concealed within the top structure 102 due to the fact that retention mechanism 162 protrudes onto the lip of cutting instrument recess 118.

In use, when the user decides to actuate the cutting instrument 114, the user can push the retention mechanism 162 inwards so that the tab of retention mechanism 162 clears the lip of cutting instrument recess 118. This can move the tab of retention mechanism 162 into the aperture of cutting instrument recess 118 so that cutting instrument 114 can advance into the aperture of cutting instrument recess 118, thus advancing the blade 116 to cut the flexible tubing. By securing cutting instrument 114 in the retracted position until an inward force moves the tab into the aperture of cutting instrument recess 118, retention mechanism 162 secures the blade 116 within top structure 102 until the user is ready to cut and therefore limits user exposure to the blade.

FIGS. 5A-5B depict in further detail how tabs 126 and catches 127 can engage with one another to secure top structure 102 to bottom structure 108. For example, tab 126B can engage with catch 127B so that the bottom ledge of tab 126B moves onto the lip of catch 127B. This engagement between the bottom ledge of tab 126B and the lip of catch 127B prevents top structure 102 from separating from bottom structure 108. For example, tab 126D of bottom structure 108 can engage with a catch (e.g., catch 127D) on the top structure 102 that is similar to the catch 127C on the opposite side of top structure 102. Each of tabs 126A-126D can engage with a respective catch of catches 127A-127D. This engagement occurs due to ledges of the tabs 126 overlapping with lips of the catches 127, thus preventing top structure 102 and bottom structure 108 from separating.

Referring now to FIGS. 5C-5D, the bottom structure 108 can include cradles 152-158. Each of cradles 152-158 can be attached to an inside surface of bottom structure 108. For example, first small cradle 152 and first large cradle 154 can be attached to an inside surface of first bottom component 110 and second large cradle 156 and second small cradle 158 can be attached to an inside surface of second bottom component 112. Cradles 152-158 extend into the open channel 113 of bottom structure 108 so that cradles 152-158 can suspend flexible tubing in a specific location within the open channel 113. As described above, first small cradle 152 and first large cradle 154 can suspend a smaller-diameter flexible tubing without interference from first large cradle 154 and second large cradle 156. Larger-diameter flexible tubing can be suspended by first large cradle 154 and second large cradle 156 with first small cradle 152 and first large cradle 154 separating to accommodate this suspension. This separating occurs when arms of first small cradle 152 separate at gap 153 and arms of second small cradle 158 separate at gap 159.

The bottom structure 108 can be placed onto flexible tubing while bottom structure 108 is separated from top structure 102, as depicted in FIGS. 5C-5D. Because open channel 113 of bottom structure 108 is completely open while bottom structure 108 is separated from top structure 102, a user can place flexible tubing within cradles 152-158 to secure bottom structure 108 onto the flexible tubing. In the example of smaller-sized flexible tubing, the user can push first small cradle 152 onto the flexible tubing so that the flexible tubing crosses an apex 164 and into a central portion of the first small cradle 152. Similarly, the user can push second small cradle 158 onto the flexible tubing so that the flexible tubing crosses an apex 166 and into a central portion of the second small cradle 158. While the flexible tubing is being pushed past apexes 164, 166, the arms of first small cradle 152 and second small cradle 158 can temporarily separate at gaps 153, 159 to accommodate the diameter of the flexible tubing. When the smaller-sized flexible tubing is secured within first small cradle 152 and second small cradle 158, the tubing can be secured to bottom structure 108. In some cases, bottom structure 108 can be slid along the flexible tubing received within first small cradle 152 and second small cradle 158 until bottom structure 108 reaches a desired location for cutting.

In the example of larger-sized flexible tubing, the user can push first large cradle 154 onto the flexible tubing so that the flexible tubing crosses an apex 166 and into a central portion of the first large cradle 154. Similarly, the user can push second large cradle 156 onto the flexible tubing so that the flexible tubing crosses an apex 169 and into a central portion of the second large cradle 156. While the flexible tubing is being pushed past apexes 168, 169, the arms of first large cradle 154 and second large cradle 156 can temporarily separate at gaps 155, 157 to accommodate the diameter of the flexible tubing. When the larger-sized flexible tubing is secured within first large cradle 154 and second large cradle 156, the tubing can be secured to bottom structure 108. In some cases, bottom structure 108 can be slid along the flexible tubing received within first large cradle 154 and second large cradle 156 until bottom structure 108 reaches a desired location for cutting. When first large cradle 154 and second large cradle 156 receive the larger-sized flexible tubing, the arms of first small cradle 152 and the second small cradle 158 can separate at gaps 153, 159 to accommodate the larger diameter of the flexible tubing. That is first large cradle 154 and second large cradle 156 can retain the larger-sized flexible tubing on the bottom structure 108, and the arms of first small cradle 152 and the second small cradle 158 can separate so that the flexible tubing passes through first small cradle 152 and the second small cradle 158 without obstruction.

Referring now to FIGS. 5E-5F, the top structure 102 of disconnector device 100 can include one or more mechanisms for closing the top structure 102 onto the bottom structure 108, securing the top structure 102 to bottom structure 108 in the closed position, retaining the cutting instrument 114 in the retracted position, and advancing the cutting instrument 114 into the top structure 102. For example, hinge posts 122 are sized to be received within hinge post receptacles 124 of bottom structure 108 so that hinge posts 122 can rotate within hinge post receptacles 124 as top structure 102 closes onto bottom structure 108. When top structure 102 fully closes onto bottom structure 108, catches 127 can engage with tabs 126 to secure top structure 102 to bottom structure 108. As discussed above, cutting instrument 114 includes a retention mechanism 162 that can prevent cutting instrument 114 from advancing into top structure 102 until the retention mechanism 162 is depressed inwards so that a tab of retention mechanism 162 no longer interfaces with a lip of cutting instrument recess 118. When the retention mechanism 162 is depressed inwards, the cutting instrument 114 can advance.

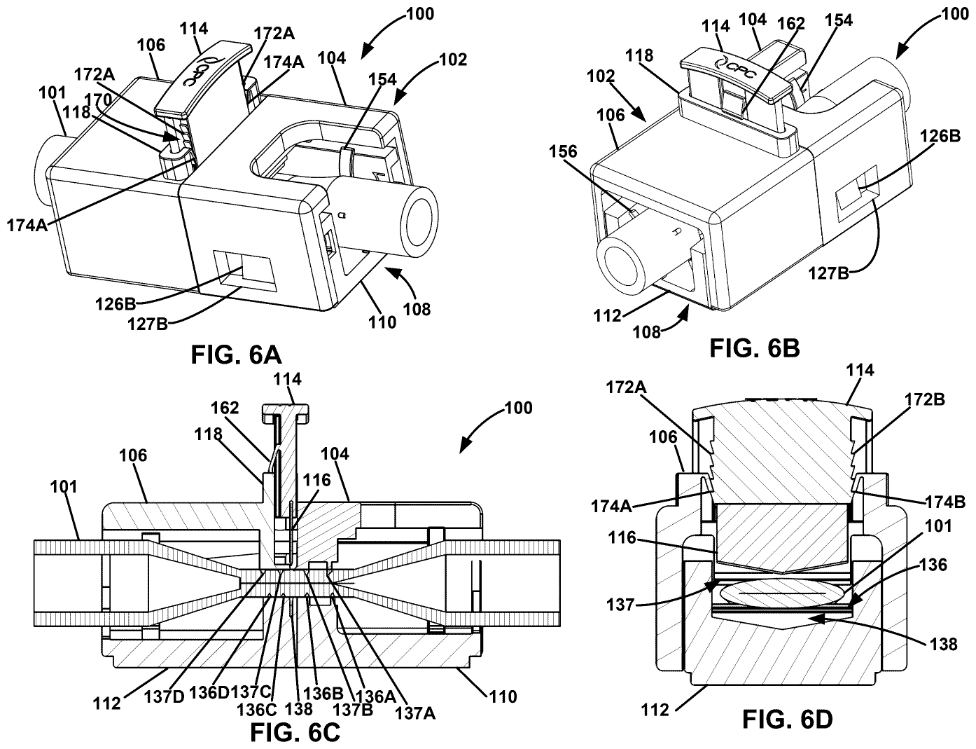

In some embodiments, disconnector device 100 includes a ratchet mechanism 170 that allows cutting instrument 114 to advance into top structure 102 and prevents cutting instrument 114 from retracting back out of top structure 102 once advancement occurs. That is, ratchet mechanism 170 can permit cutting instrument 114 to move in a first direction while preventing cutting instrument 114 from moving in a second direction opposite the first direction. This movement limitation serves to prevent cutting instrument 114 from freely moving between the actuated position and the retracted position and ensures that the blade 116 is always secured in a safe place without being exposed to users. For example, when the user depresses retention mechanism 162 and advances the cutting instrument 114 from the retracted position to the actuated position, the ratchet mechanism 170 can maintain the cutting instrument 114 in the actuated position where the blade 116 is housed within a protective blade recess 138 in the bottom structure 108. Consequently, retention mechanism 162 can maintain blade 116 within the top structure 102 in the retracted position as depicted in FIG. 6B, and ratchet mechanism 170 can maintain blade 116 within the protective blade recess 138 in the bottom structure 108. Retention mechanism 162 and ratchet mechanism 170 therefore ensure that blade 116 is not exposed to users in the retracted position and the actuated position.

FIGS. 5G-5I depict three different stages of attachment between top structure 102 and bottom structure 108 of disconnector device 100. For example, FIG. 5G depicts a first stage where top structure 102 is completely separated from bottom structure 108 and top structure 102 is aligned perpendicular to bottom structure 108. This means that hinge post 122B is oriented so that its short dimension extends vertically to slide into hinge post receptacle 124B. Tab 125 can secure hinge post 122B in place once hinge post 122B fully slides into hinge post receptacle 124B. Another hinge post (e.g., hinge post 122A) of top structure 102 can be oriented to slide into another hinge post receptacle (e.g., hinge post receptacle 124A) of bottom structure 108, with another hinge tab securing hinge post receptacle 124A in place. FIG. 5H depicts a second stage where hinge post 122B of top structure 102 is partially inserted into hinge post receptacle 124B. FIG. 5I depicts a third stage where hinge post 122B is fully inserted into hinge post receptacle 124A with tab 125 securing the hinge post and hinge post receptacle together.

As depicted in FIG. 6B, ratchet mechanism 170 includes ratchet tabs 172A on a first side of cutting instrument 114 and ratchet tabs 172B on a second side of cutting instrument 114. Each ratchet tab includes a sloped portion and an upward-facing ledge. Ratchet mechanism 170 also includes a bendable ratchet lip 174A on the first side of cutting instrument 114 and a bendable ratchet lip 174B on the second side of cutting instrument 114. Ratchet tabs 172A can engage with bendable ratchet lip 174A and ratchet tabs 172B can engage with bendable ratchet lip 174B to allow cutting instrument 114 to advance into top structure 102 in a first direction while preventing cutting instrument 114 from moving in a second direction opposite the first direction.

For example, in the retracted position, the bendable ratchet lip 174A engages with the ledge of a first one of ratchet tabs 172A and the bendable ratchet lip 174B engages with the ledge of a first one of ratchet tabs 172B. As the cutting instrument 114 advances into top structure 102, the sloped portion of the second one of ratchet tabs 172A pushes the bendable ratchet lip 174A inwards until the bendable ratchet lip 174A engages with the ledge of the second one of ratchet tabs 172A. Similarly, the sloped portion of the second one of ratchet tabs 172B pushes the bendable ratchet lip 174B inwards until the bendable ratchet lip 174B engages with the ledge of the second one of ratchet tabs 172B. This process repeats until bendable ratchet lip 174A engages with ledge of a last one of ratchet tabs 172A and bendable ratchet lip 174B engages with a ledge of a last one of ratchet tabs 172B. This corresponds to the actuated position, where the cutting instrument 114 is fully extended into the top structure 102.

In some embodiments, ratchet mechanism 170 only allows cutting instrument 114 to move in one direction while preventing movement in the opposite direction because the sloped portions of ratchet tabs 172A and 172B are oriented to bend the bendable ratchet lips 174A and 174B only when cutting instrument 114 advances into top structure 102. Bendable ratchet lips 174A and 174B engage with the ledges of ratchet tabs 172A and 172B in a way that prevents outward movement of cutting instrument 114. This means that ratchet mechanism 170 can secure the cutting instrument 114 in the actuated position after the flexible tubing is cut, without allowing the blade 116 to dislodge from the protective blade recess 138.

Referring now to FIGS. 6A-6D, when top structure 102 closes onto bottom structure 108, the tabs 126 can engage with catches 127 to secure top structure 102 to bottom structure 108 to form an integrated disconnector device 100. Although the interaction between tab 126B and catch 127B is the only tab/catch interaction depicted in FIGS. 6A-6D, each of tabs 126A-126D can engage with a respective catch of catches 127A-127D to secure top structure 102 to bottom structure 108. In some embodiments, tabs 126 and catches 127 can permanently secure top structure 102 to bottom structure 108 once top structure 102 fully closes onto bottom structure 108.

When top structure 102 closes onto bottom structure 108, pinching teeth 136, 137 can compress flexible tubing 101 within the disconnector device 100 such that the lumen of flexible tubing 101 in the compressed area is completely closed, as depicted in FIGS. 6C-6D. This prevents fluid from crossing the compressed area between the left side of the compression and the right side of the compression. In some cases, pinching teeth 136, 137 provide a balance between a small surface area to limit force necessary to compress flexible tubing 101 and retention capability. For example, a surface area of pinching teeth 136, 137 that interfaces with flexible tubing 101 when top structure 102 is secured to bottom structure 108 is small so that a user can manually compress flexible tubing 101 by closing top structure 102 onto bottom structure 108, using hinge posts 122 and hinge post receptacles 124 as a fulcrum point for leverage. Additionally, as depicted in FIG. 6C, pinching teeth 136, 137 each include a pointed protrusion that can press into the surface of flexible tubing 101, thus retaining flexible tubing 101 in place relative to disconnector device 100.

Placing two pairs of pinching teeth on each side of disconnector device 100 can strike a healthy balance where the compression surface area is small enough so that a user can manually close top structure 102 onto bottom structure 108 and the pinching teeth are numerous enough to hold the disconnector device 100 in place on flexible tubing 101 before and after separation. For example, a one side of disconnector device 100 includes pinching teeth 136A, 136B, 137A, and 137B and another side of disconnector device 100 includes pinching teeth 136C, 136D, 137C, and 137D. This ensures that even when disconnector device 100 separates into two parts after the flexible tubing 101 is cut, two pairs of pinching teeth maintain compression on one side of the cut and two pairs of pinching teeth maintain compression on another side of the cut.

As depicted in FIGS. 6A-6D, cutting instrument 114 can occupy the retracted position after top structure 102 is fully closed onto bottom structure 108 and before the blade 116 cuts through flexible tubing 101. This ensures that disconnector device 100 can fully compress the flexible tubing 101 before cutting the flexible tubing 101 in the compressed region. This means that when the flexible tubing 101 is eventually cut, both sides of the cut are compressed to completely close the lumen in such a way that fluid cannot escape through the cut after it is made and contamination cannot enter the cut after it is made. In the retracted position, a sharp edge of blade 116 rests above the flexible tubing 101, displaced from the flexible tubing 101 by a distance. Retention mechanism 162 can maintain the cutting instrument 114 in the retracted position until the user presses the retention mechanism 162 inwards past the lip of cutting instrument recess 118. FIG. 6C, for example, depicts the retention mechanism 162 overlapping with the lip of cutting instrument recess 118 in a way that prevents cutting instrument 114 from prematurely advancing into disconnector device 100.

Second bottom component 112 can define a protective blade recess 138 for receiving the blade 116 when cutting instrument 114 transitions from the retracted position to the actuated position. For example, protective blade recess 138 can be sized to completely receive a sharp end of blade 116 so that blade 116 is not exposed to a user when cutting instrument 114 transitions from the retracted position to the actuated position. In the example depicted in FIG. 6D, blade 116 has a triangular-shaped sharp edge that defines a point. This point can be advantageous for piercing the cutting instrument 114. For example, when a user advances cutting instrument 114 downwards into disconnector device 100, the force is concentrated on the pointed end of blade 116, making it easy for blade 116 to pierce the flexible tubing 101. Blade 116 is not limited to having the shape depicted in FIG. 6D. Other shapes are also possible for blade 116, including flat blades, angled blades, and beveled blades.

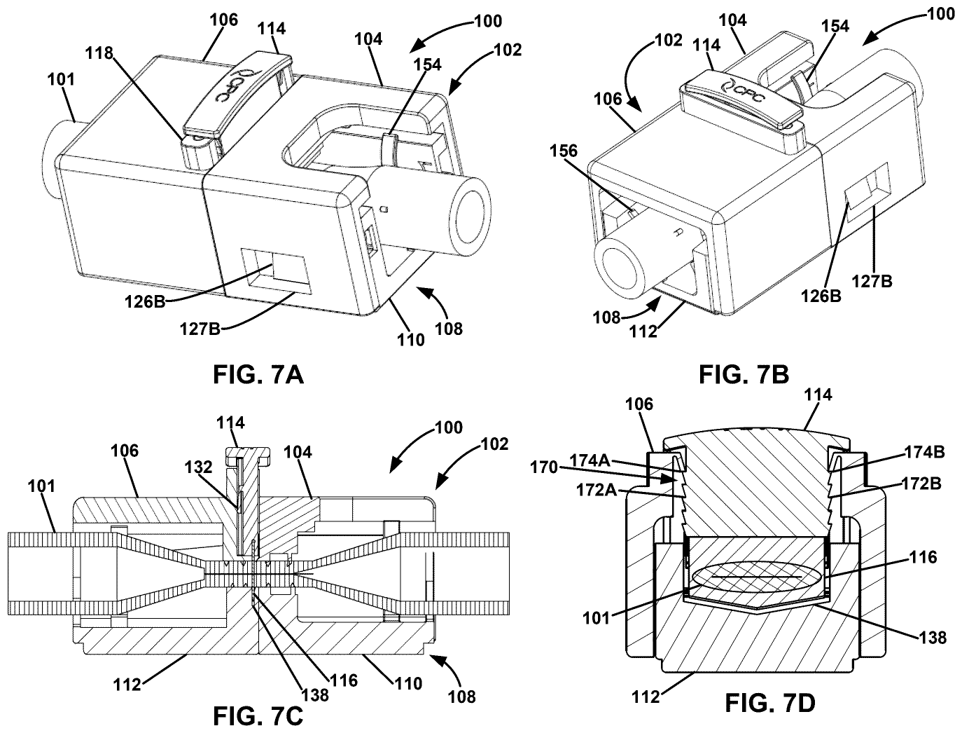

Referring now to FIGS. 7A-7D, cutting instrument 114 can transition from the retracted position to an actuated position to cut completely through flexible tubing 101. As discussed above, to advance cutting instrument 114, a user can press the retention mechanism 162 inwards until the retention mechanism 162 clears the lip of cutting instrument recess 118. This allows cutting instrument 114 to advance into disconnector device 100 so that blade 116 cuts through flexible tubing 101. When cutting instrument 114 reaches the actuated position, blade 116 has cut completely through a cross-section of the compressed flexible tubing 101 as depicted in FIG. 7B. Compression force on both sides of the cut prevents potentially biohazardous liquid from leaking out of the cut. For example, pinching teeth 136A, 136B and pinching teeth 137A, 137B can maintain compression force on one side of the cut and pinching teeth 136C, 136D and pinching teeth 137C, 137D can maintain compression force another side of the cut. As depicted in FIGS. 7C-7D, blade 116 can be housed within protective blade recess 138 when the blade 116 is in the actuated position.

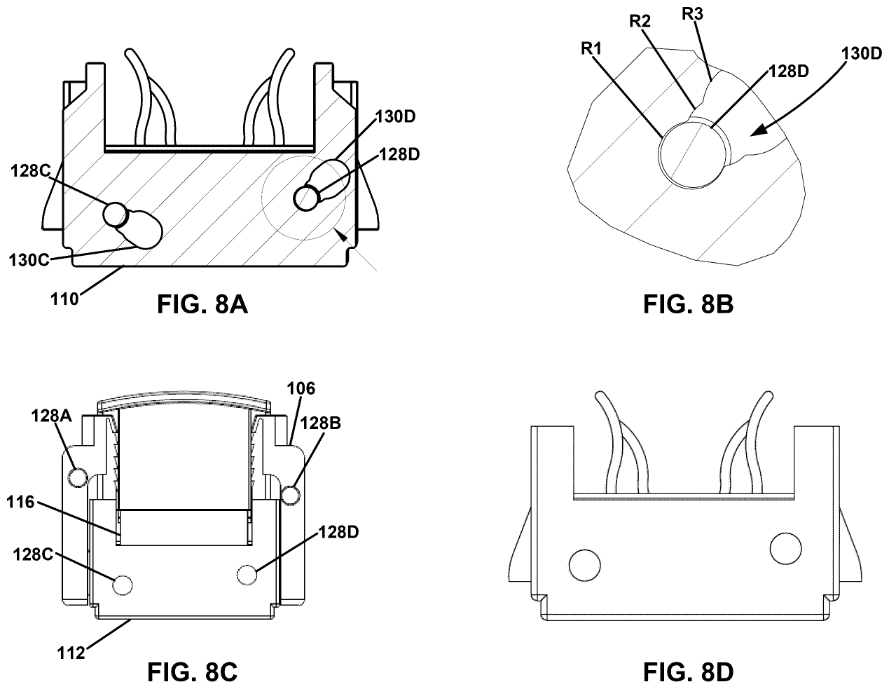

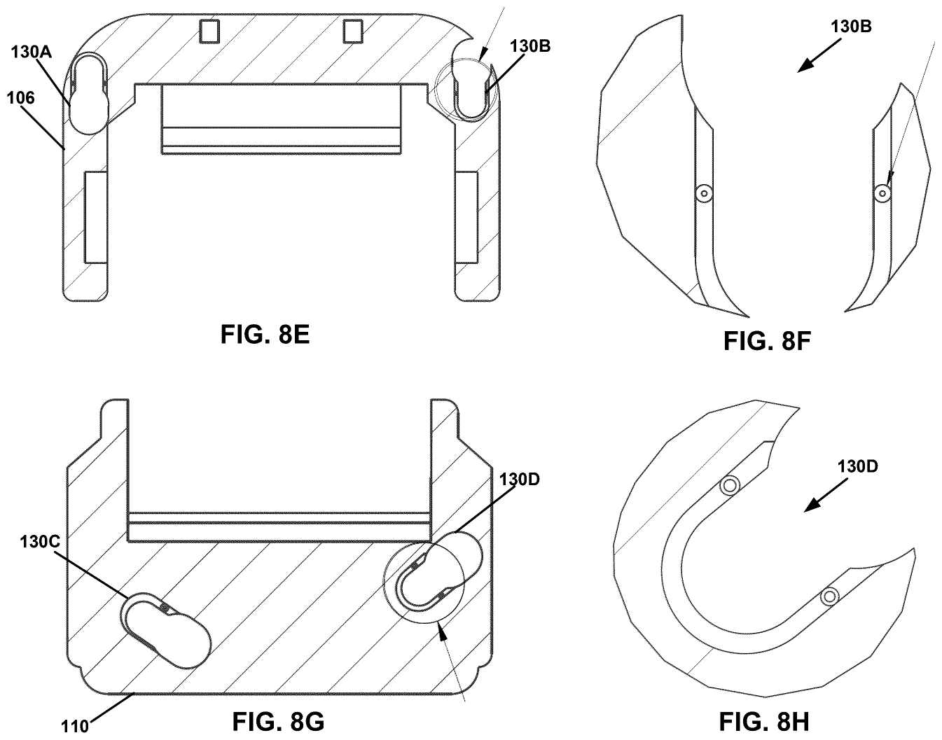

FIGS. 8A-8D depict embodiments for how twist disconnect posts 128 can engage with twist disconnect receptacles 130. As described above, each of twist disconnect posts 128A-128D can engage with a twist disconnect receptacle 130A-120D. In some examples, each twist disconnect receptacle 130 can include three different sections (e.g., “R1,” “R2,” and “R3” of FIG. 8B). These three different sections can represent three different diameters, with the smallest-diameter section being the section that secures a twist disconnect post of twist disconnect posts 128. Twist disconnect posts 128 can disengage from twist disconnect receptacle 130 by rotating relative to twist disconnect receptacles 130. In the example of FIG. 8B, such rotation can cause twist disconnect posts 128D to dislodge from the “R1” section of twist disconnect receptacle 130D and enter the larger sections of twist disconnect receptacle 130D. The other twist disconnect posts 128 can perform a similar dislodgement from twist disconnect receptacles 130 so that all of twist disconnect posts 128 dislodge from the smallest diameter section of twist disconnect receptacles 130 simultaneously.

FIGS. 8E-8H depict another embodiment of twist disconnect receptacles 130. For example, second top component 106 can define twist disconnect receptacles 130A, 130B and second bottom component 110 can define twist disconnect receptacles 130C, 130D. In the embodiment of FIGS. 8E-8H, each of twist disconnect receptacles 130 include two sections instead of the three sections depicted in FIGS. 8A-8B. These two sections of twist disconnect receptacles 130 depicted in FIGS. 8E-8H include a smaller section for retaining twist disconnect posts 128 and a larger section where twist disconnect posts 128 can dislodge to.

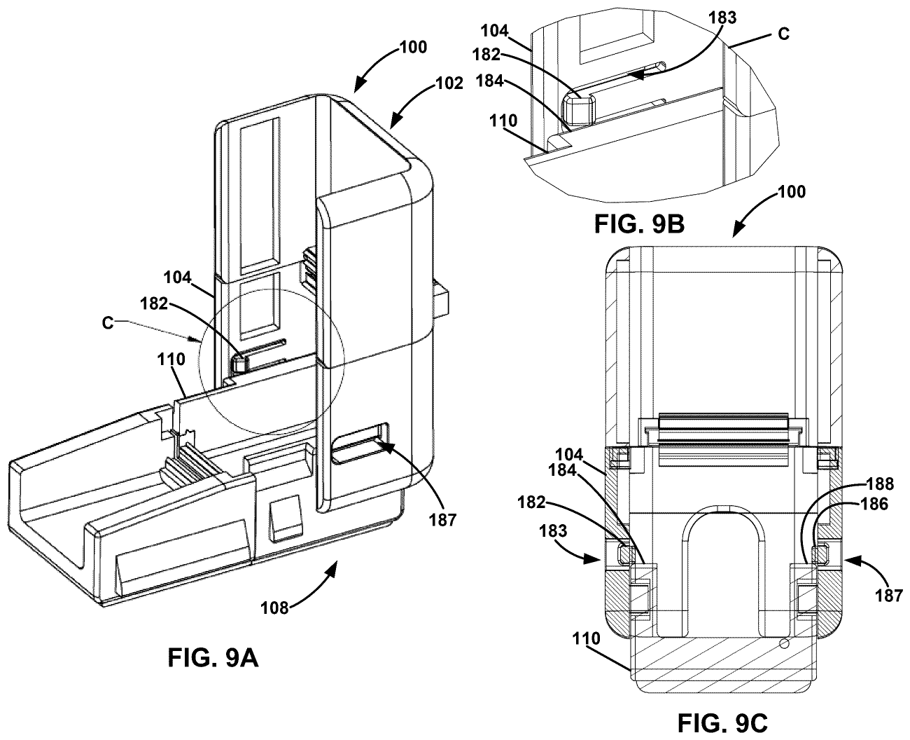

FIGS. 9A-13C depict embodiments of a kickstand feature for supporting top structure 102 in an upright position relative to bottom structure 108 when hinge posts 122 are inserted into hinge post receptacles 124. In some cases, it is beneficial to prevent top structure 102 in from closing onto bottom structure 108 until the user has positioned disconnector device 100 where the user desires to cut the flexible tubing. This is because in some cases, top structure 102 cannot separate from bottom structure 108 once top structure 102 closes onto bottom structure 108 and tabs 126 engage with catches 127. That is, prematurely closing top structure 102 onto bottom structure 108 could render disconnector device 100 unusable or cause disconnector device 100 to compress the flexible tubing in the wrong place. The embodiments depicted in FIGS. 9A-13C provide mechanisms to keep top structure 102 in the open position relative to bottom structure 108 until the user elects to close top structure 102 onto bottom structure 108.

Referring now to FIGS. 9A-9C, disconnector device 100 can include an embodiment of a kickstand mechanism where first top component 104 defines a first protruding element 182 and a second protruding element 186. First protruding element 182 is configured to extend over a first ledge 184 of first bottom component 110 in a way that prevents top structure 102 from closing onto bottom structure 108. Similarly, second protruding element 186 can extend over a second ledge 188 of first bottom component 110 in a way that resists top structure 102 closing onto bottom structure 108. Because at least part of first protruding element 182 extends over first ledge 184 and at least part of second protruding element 186 extends over second ledge 188, first protruding element 182 and second protruding element 186 prevent top structure 102 from closing onto bottom structure 108.

In the embodiment of FIGS. 9A-9C, first bottom component 110 defines a first aperture 183 corresponding to first protruding element 182 and a second aperture 187 corresponding to second protruding element 186. To close top structure 102 from closing onto bottom structure 108, first protruding element 182 can be pushed into first aperture 183 and second protruding element 186 can be pushed into second aperture 187 so that first protruding element 182 no longer extends over first ledge 184 and second protruding element 186 no longer extends over second ledge 188. This allows the outer casing of top structure 102 to close onto the outer casing of bottom structure 108 without further resistance from first protruding element 182 and second protruding element 186. In some cases, the user can disengage first protruding element 182 and second protruding element 186 by manually pushing first protruding element 182 and second protruding element 186 into the respective apertures. In some cases, first protruding element 182 and second protruding element 186 automatically retreat into first aperture 183 and second aperture 187 when a great enough force is applied to rotate top structure 102 relative to bottom structure 108.

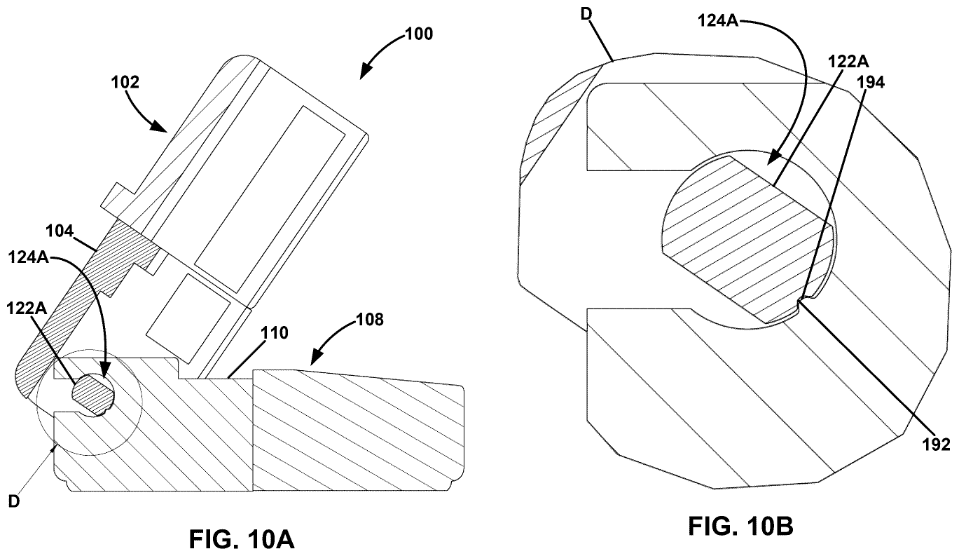

FIGS. 10A-10B depict another kickstand embodiment for maintaining top structure 102 in an upright position relative to bottom structure 108. As discussed previously, first top component 104 of top structure 102 can define hinge posts 122 and first bottom component 110 can define hinge post receptacles 124 for receiving hinge posts 122. In some examples, hinge post 122A can be received within hinge post receptacle 124A and hinge post 122B can be received within hinge post receptacle 124B. to maintain top structure 102 in the upright position relative to bottom structure 108 as depicted in FIG. 10A, hinge post 122A can define a detent 192 and hinge post receptacle 124A can define a protrusion 194 that engages with the detent 192. For example, when protrusion 194 engages with the detent 192, this resists top structure 102 rotating onto bottom structure 108.

Top structure 102 can rotate relative to bottom structure 108 in a way that causes hinge post 122A to rotate clockwise to disengage detent 192 from protrusion 194. This can be achieved by the user applying more than a threshold amount of rotational force to hinge post 122A. In some embodiments, gravity alone is not strong enough to cause top structure 102 to rotate downwards onto bottom structure 108. This means that disconnector device 100 can sit idle on the flexible tubing without top structure 102 closing onto bottom structure 108 prematurely. In some examples, hinge post 122B and hinge post receptacle 124B on the opposite side of disconnector device 100 from the perspective of FIGS. 10A-10B. This can provide further resistance against premature closure.

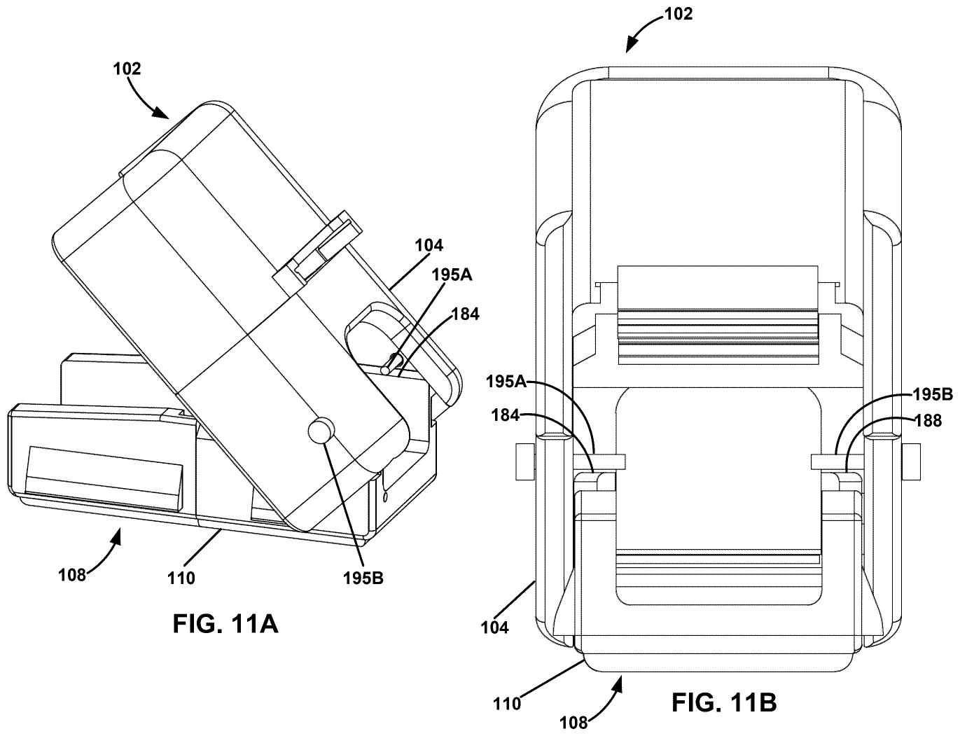

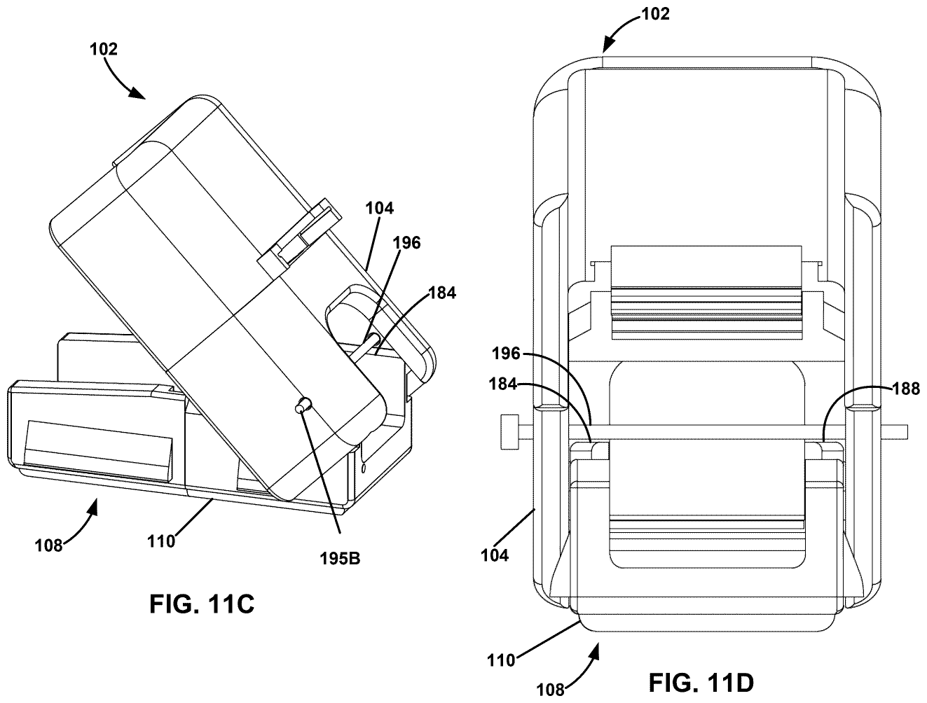

Referring now to FIGS. 11A-11B, another kickstand embodiment for maintaining top structure 102 in an upright position relative to bottom structure 108 involves using kickstand pins 195A-195B, which prevent top structure 102 from closing onto bottom structure 108, even when force is applied to close top structure 102 onto bottom structure 108. For example, kickstand pin 195A can extend through an aperture of the first top component 104 and onto a first ledge 184 of first bottom component 110. Because kickstand pin 195A is positioned this way, kickstand pin 195A prevents top structure 102 from closing onto bottom structure 108 past the position depicted in FIG. 11A. In some cases, disconnector device 100 also includes kickstand pin 195B which extends through an aperture of the first top component 104 and onto a second ledge 188 of first bottom component 110. Kickstand pin 195B thus likewise prevents top structure 102 from closing onto bottom structure 108 past the position depicted in FIG. 11A. To close the top structure 102 onto bottom structure 108, the user can remove kickstand pin 195A and kickstand pin 195B from their respective apertures, thus allowing top structure 102 to rotate beyond the position depicted in FIG. 11A.

FIGS. 11C-11D depict another kickstand embodiment where a single kickstand pin passes through a first aperture of first top component 104 and a second aperture of first top component 104 to cross both the first ledge 184 of first bottom component 110 and the second ledge 188 of the first bottom component 110. Kickstand pin 196 prevents top structure 102 from closing onto bottom structure 108 beyond the position depicted in FIG. 11C. To allow top structure 102 to close onto bottom structure 108, a user can remove kickstand pin 196 from the first aperture and the second aperture in first top component 104.

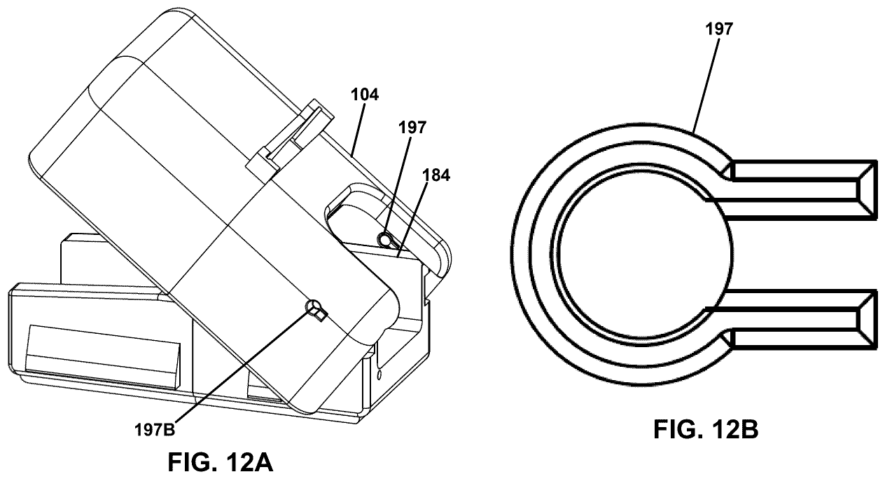

FIGS. 12A-12B depict another kickstand embodiment where a raised tab 197 on an inner surface of first top component 104 rests on first ledge 184 of first bottom component 110 to resist top structure 102 from closing onto bottom structure 108 beyond the position depicted in FIG. 11A. In some cases, another raised tab can be located in the same position on an opposite side of first top component 104. In this embodiment, top structure 102 can close onto bottom structure 108 when enough force is applied to move raised tab 197 beyond first ledge 184. FIG. 12B depicts a close-up view of raised tab 197.

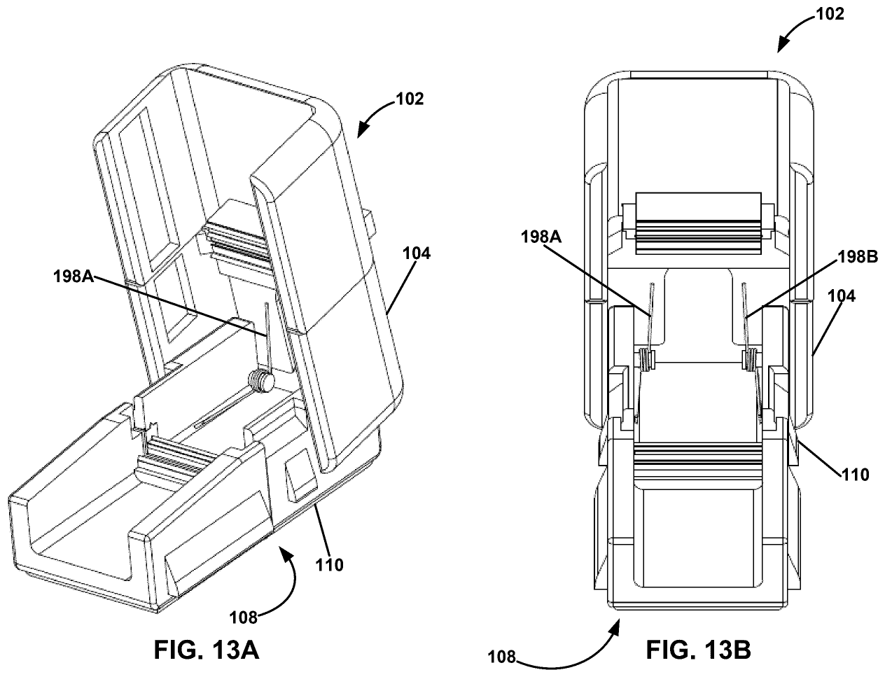

Referring now to FIGS. 13A-13B, another kickstand embodiment involves spring support members 198A-198B which can maintain top structure 102 in a raised position relative to bottom structure 108. These support members 198A-198B each include a first component which rests on an inside surface of first top component 104, a second component which rests on an inside surface of first bottom component 110, and a spring component which applies force against the first top component 104 and first bottom component 110. Consequently, support members 198A-198B can apply force which resists top structure 102 from closing onto bottom structure 108.

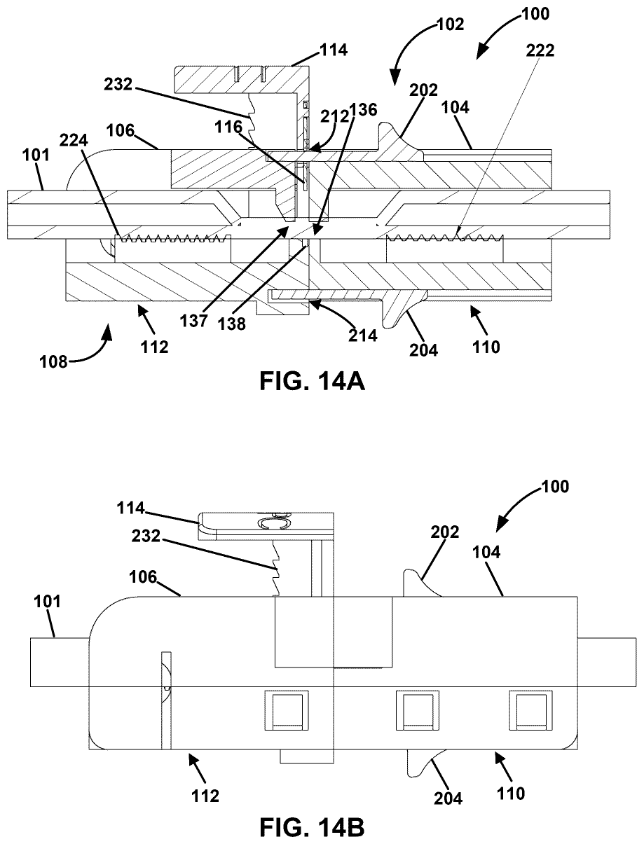

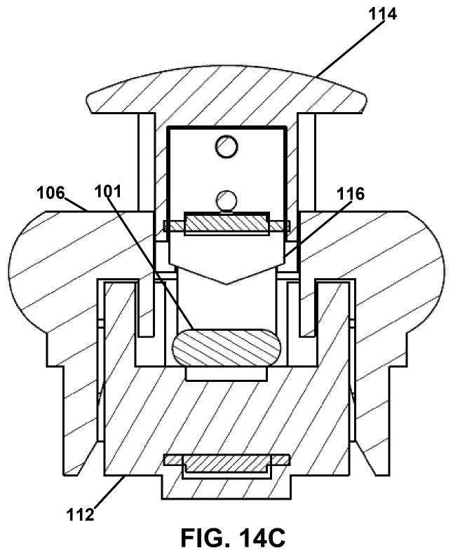

FIGS. 14A-14C depict an embodiment of disconnector device 100 where a sliding mechanism 202 secures the cutting instrument 114 in the retracted position before the blade 116 cuts through flexible tubing 101. For example, as depicted in FIG. 14A, the sliding mechanism 202 can pass through an aperture 212 in the cutting instrument 114 such that sliding mechanism 202 prevents cutting instrument 114 from advancing into the outer housing of disconnector device 100. When sliding mechanism 202 passes through aperture 212, the overlap between sliding mechanism 202 and a ledge of the aperture 212 prevents movement of cutting instrument 114 from the retracted position depicted in FIG. 14A. It can be beneficial for cutting instrument 114 to remain in the retracted position until disconnector device 100 is positioned to cut the flexible tubing 101 so that cutting instrument 114 does not prematurely transition to the actuated state where blade 116 is exposed in an unsafe manner or the tubing is cut prematurely. By maintaining the cutting instrument 114 in the retracted position, blade 116 remains safely housed within top structure 102.

In some cases, sliding mechanism 202 is slidably attached to a surface of first top component 104 such that sliding mechanism 202 can occupy an unlocked position where sliding mechanism 202 passes through aperture 212 and an unlocked position where sliding mechanism 202 does not pass through aperture 212. To transition between the locked position and the unlocked position, sliding mechanism 202 can slide laterally along a longitudinal axis of disconnector device 100 from the perspective of FIG. 14A. For example, sliding mechanism 202 “unlocks” by sliding rightwards out of the aperture 212 and “locks” by sliding leftwards into the aperture 212.

In addition to locking cutting instrument 114 in the retracted position, sliding mechanism 202 can also secure second top component 106 to first top component 104 by preventing second top component 106 from rotating relative to first top component 104. The sliding mechanism 202 can, in some examples, engage with one or more grooves of the first top component 104 and second top component 106, and sliding mechanism 202 can traverse an interface between first top component 104 and second top component 106. Sliding mechanism 202 therefore can prevent rotation of second top component 106 relative to first top component 104 because sliding mechanism 202 connects first top component 104 and second top component 106 across an interface between these two components.

The embodiment of disconnector device 100 can, in some embodiments, include a sliding mechanism 204 attached to the bottom surface of first bottom component 110 opposite sliding mechanism 202 which is attached to the top surface of first top component 104. Sliding mechanism 204 can occupy a locked position where sliding mechanism 204 is received within an aperture 214 in the second bottom component 112, as depicted in FIG. 14A. When sliding mechanism 204 is received within an aperture 214, the sliding mechanism can engage with one or more inner walls of the aperture 214 such that sliding mechanism 204 connects the first bottom component 110 and second bottom component 112. Sliding mechanism 204 can therefore prevent second bottom component 112 from rotating relative to first bottom component 110. When one or both of sliding mechanisms 202, 204 occupy the locked position as depicted in FIG. 14A, sliding mechanisms 202, 204 prevent first bottom component 110 and first top component 104 from rotating relative to second bottom component 112 and second top component 106.

In some embodiments, disconnector device 100 can include retention mechanisms 222, 224 for retaining flexible tubing 101 in place within disconnector device 100. For example, disconnector device 100 includes pinching teeth 136 and 137 which can compress flexible tubing 101 and retain flexible tubing 101 within disconnector device 100. Retention mechanisms 222, 224 can serve to provide further retention of flexible tubing 101 in addition to the retention provided by pinching teeth 136 and 137. As depicted in FIG. 2A, retention mechanism 222 can be attached to an inner surface of first bottom component 110 and retention mechanism 225 can be attached to an inner surface of second bottom portion 112. Retention mechanisms 222, 224 each include teeth which apply a friction force to the surface of flexible tubing 101, thus resisting lateral movement of flexible tubing 101 within disconnector device 100.

Retention mechanisms 222, 224 can be positioned within disconnector device 100 so that there are no paired retention mechanisms extending from the top inner surfaces of disconnector device 100. This means that flexible tubing 101 can rest on retention mechanisms 222, 224 without being compressed by retention mechanisms 222, 224. This can differentiate retention mechanisms 222, 224 from pinching teeth 136 and 137 which do compress flexible tubing 101 in some embodiments. As depicted in FIG. 14A, pinching teeth 136 and 137 in the center of disconnector device 100 compress the flexible tubing 101 using a small compression surface area, with flexible tubing 101 resting on retention mechanisms 222, 224 having a significantly larger surface area. Because retention mechanisms 222, 224 retain without compressing, the retention mechanisms 222, 224 can provide added retention without significantly increasing the force required to compress flexible tubing 101 with pinching teeth 136 and 137.

FIG. 14C depicts a cross-section of disconnector device 100 with cutting instrument 114 in the retracted position before blade 116 cuts the flexible tubing 101. In the position depicted in FIG. 14C, the second top component 106 is closed completely over second bottom component 112 so that pinching teeth 136 and 137 are compress flexible tubing 101. When flexible tubing 101 is compressed, the blade 116 can be positioned to cut flexible tubing 101 when cutting instrument 114 transitions from the retracted position to the actuated position. This transition causes the blade 116 to cut completely through flexible tubing 101 in a location where flexible tubing 101 is compressed by pinching teeth 136 and 137. Ratchet mechanism 232 is shaped to permit cutting instrument 114 to advance into disconnector device 100 while preventing cutting instrument 114 from retracting out of disconnector device 100.

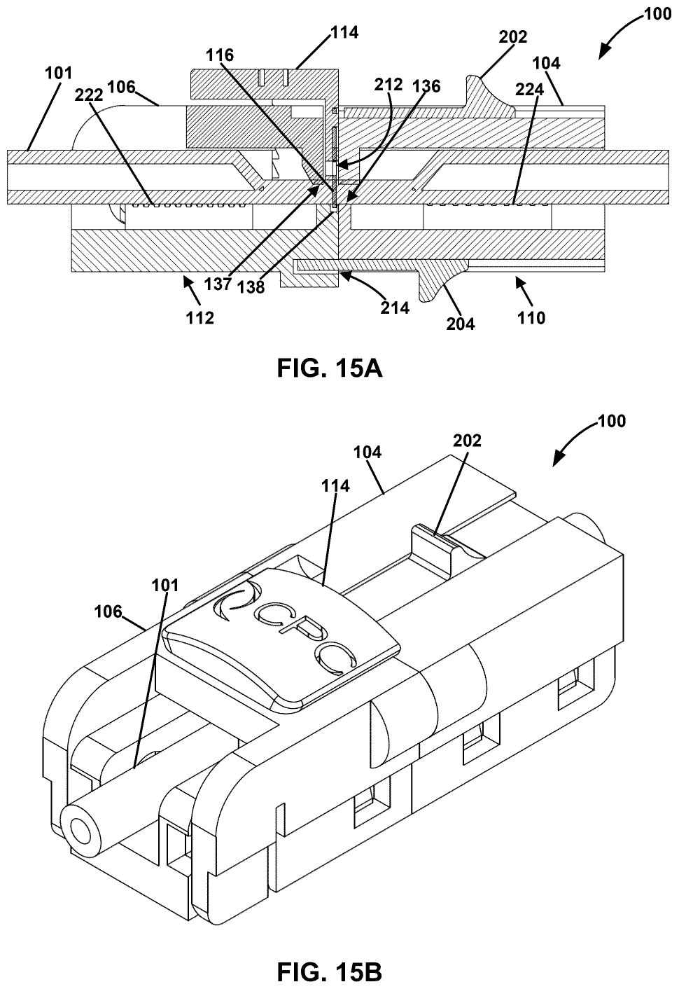

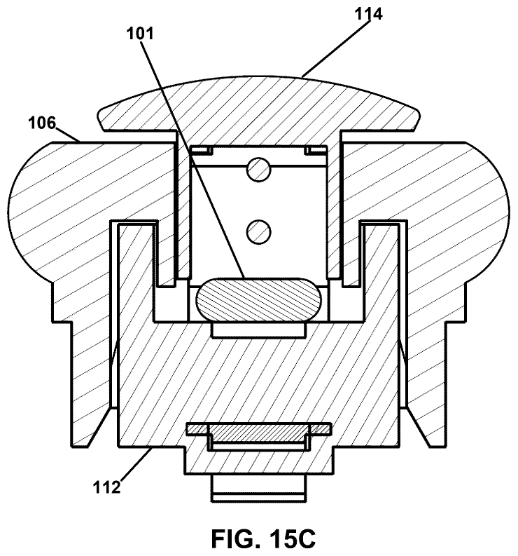

Referring now to FIGS. 15A-15C, cutting instrument 114 can advance into disconnector device 100 to cut flexible tubing 101 in a location where flexible tubing 101 is compressed by pinching teeth 136 and 137. For example, in some embodiments, blade 116 cuts completely through flexible tubing 101 so that blade 116 enters protective blade recess 138. Before cutting instrument 114 advances into disconnector device 100, sliding mechanism 202 transitions from the locked position depicted in FIG. 14A where sliding mechanism 202 passes through aperture 212 of cutting instrument 114 to the unlocked position depicted in FIG. 15A where sliding mechanism 202 is removed from aperture 212 to allow movement of cutting instrument 114. In some examples, sliding mechanism 202 transitions from the locked position to the unlocked position by sliding rightwards along the surface of first top component 104.

As depicted in FIG. 15A, sliding mechanism 204 can remain in the locked position while sliding mechanism 202 occupies the unlocked position such that sliding mechanism 204 prevents rotation of first top component 104 and first bottom component 110 relative to second top component 106 and second bottom component 112. To rotate first top component 104 and first bottom component 110 relative to second top component 106 and second bottom component 112, the user can transition first top component 104 from the locked position to the unlocked position. Moving the sliding mechanism 202 from the locked position to the unlocked position allows twist disconnect posts to disengage from twist disconnect receptacles, thus allowing second top component 106 and second bottom component 112 to separate from first top component 104 and first bottom component 110. FIG. 15C depicts a cross-section of disconnector device 100 when cutting instrument 114 is in the actuated state, cutting through flexible tubing 101.

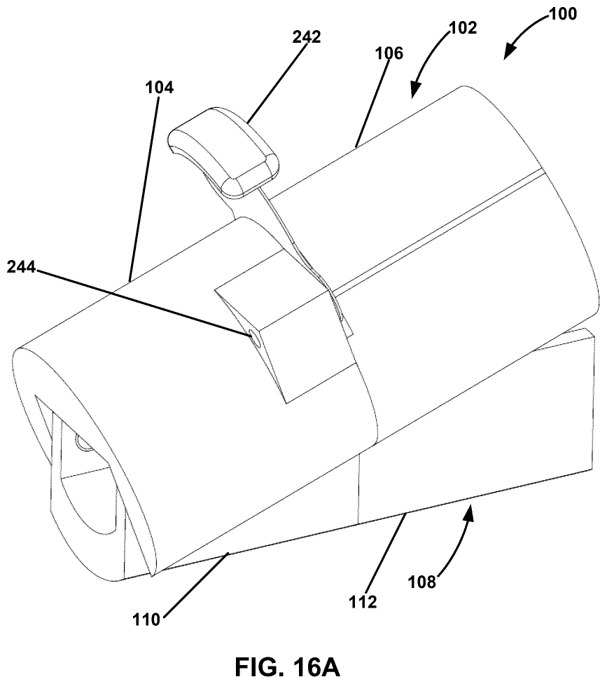

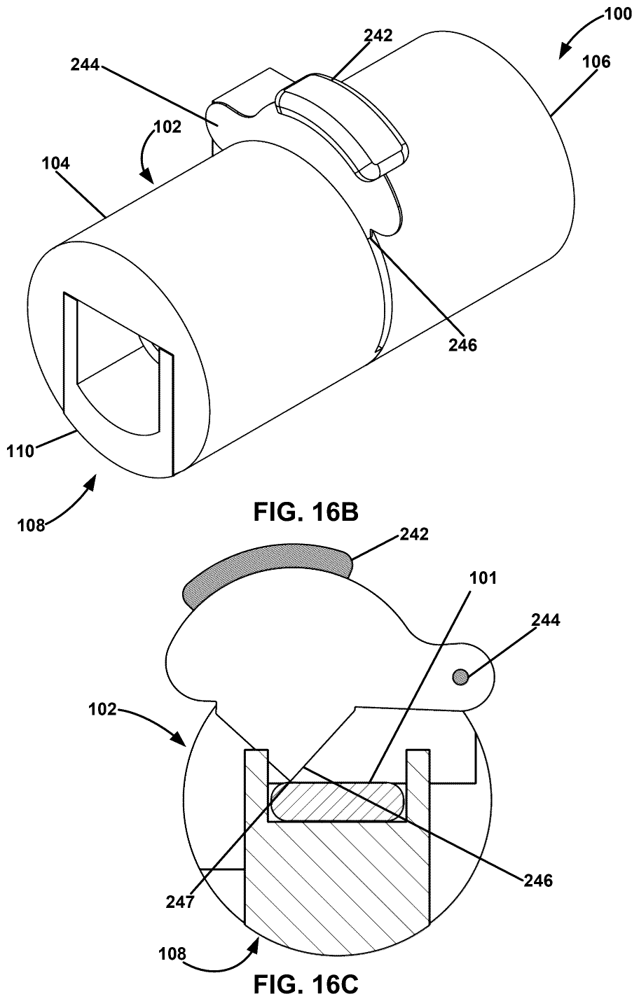

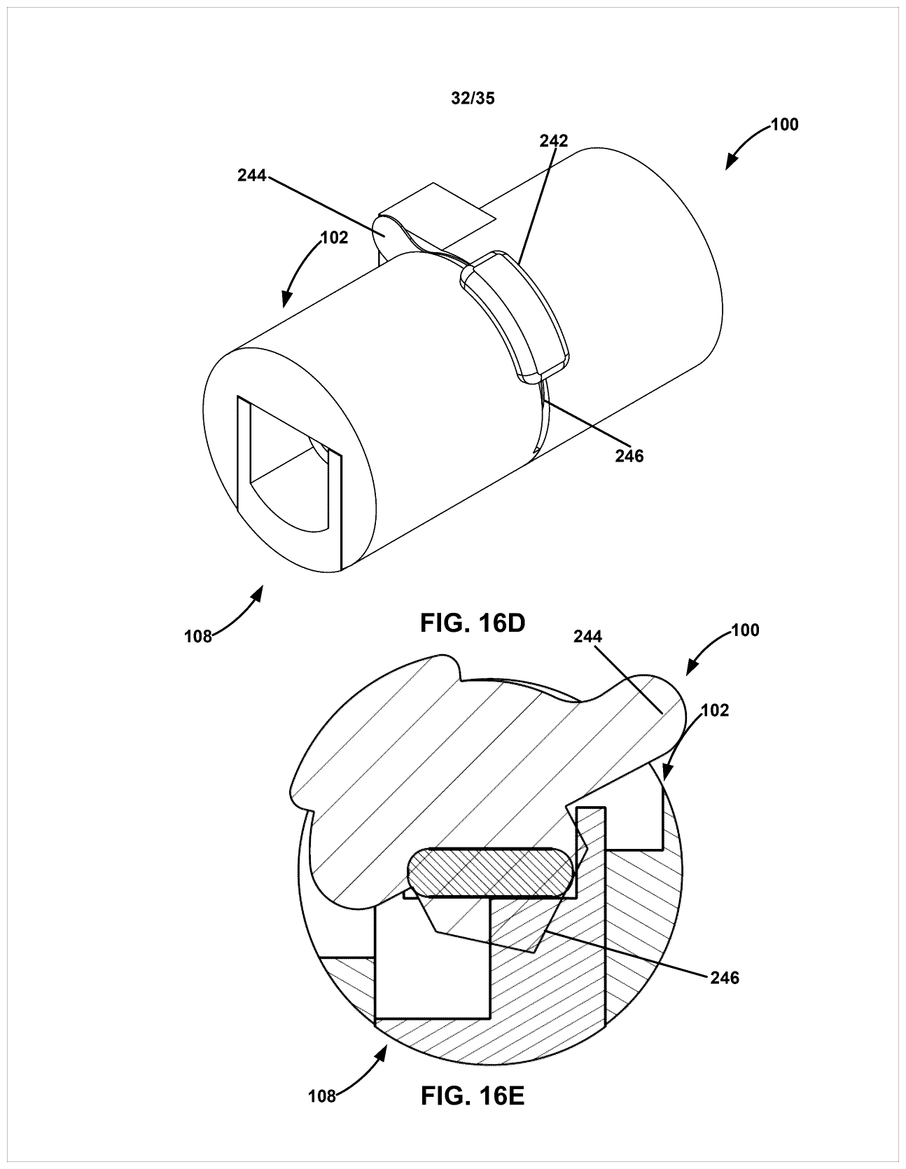

FIGS. 16A-16E depict an embodiment of disconnector device 100 where a profile of the disconnector device 100 is round and a cutting mechanism 242 rotates about a hinge instead of advancing in a linear fashion. For example, disconnector device 100 in the embodiment of FIGS. 16A-16C includes a top structure 102 that can close on top of a bottom structure 108 to compress flexible tubing at a compression zone. When top structure 102 fully closes onto bottom structure 108, the first top component 104 and the first bottom component 110 form a first connected unit and the second top component 106 and the second bottom component 112 form a second connected unit. The first connected unit and the second connected unit can disconnect from each other when first top component 104 and the first bottom component 110 rotate relative to second top component 106 and the second bottom component 112.

Top structure 102 can fully close onto bottom structure 108 so that top structure 102 and bottom structure 108 compress flexible tubing 101 at a compression zone. When top structure 102 and bottom structure 108 are fully closed together to compress flexible tubing 101, cutting mechanism 242 can be positioned to cut through flexible tubing 101 by transitioning from a retracted position to an actuated position. In FIGS. 16A-16C, cutting mechanism 242 is in a retracted position where a portion of blade 246 is outside of the outer housing of disconnector device 100. Specifically, point 247 of the blade 246 can rest on or near a surface of flexible tubing 101 before cutting through flexible tubing 101. To transition between the retracted position and the actuated position, cutting mechanism 242 can rotate about hinge 244 to advance into disconnector device 100 and through the flexible tubing 101. For example, cutting mechanism 242 can transition from the retracted position depicted in FIGS. 16B-16C to the actuated position depicted in FIGS. 16D-16E. In the actuated position, blade 246 cuts completely through flexible tubing 101.

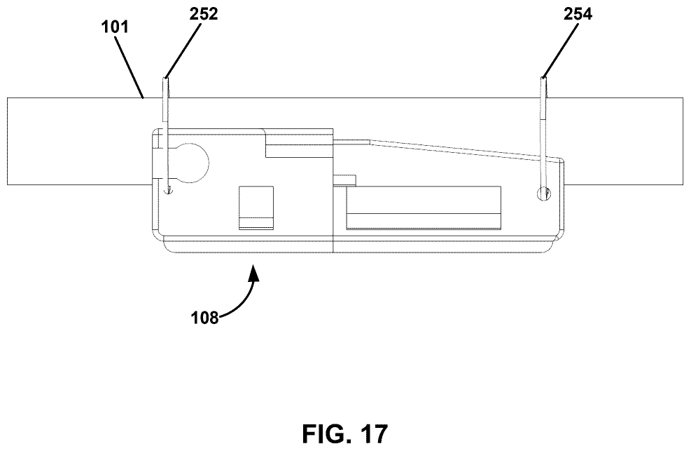

FIG. 17 depicts an embodiment where bottom structure 108 can be connected to flexible tubing 101 by connectors 252, 254. These connectors 252, 254 can extend through apertures in bottom structure 108 and over flexible tubing 101 to secure bottom structure 108 to the flexible tubing 101 before top structure 102 connects to bottom structure 108. In some cases, tubing can be shipped already connected to bottom structure 108. Top structure 102 can connect to bottom structure 108 to cut the flexible tubing 101 when desired by a user.

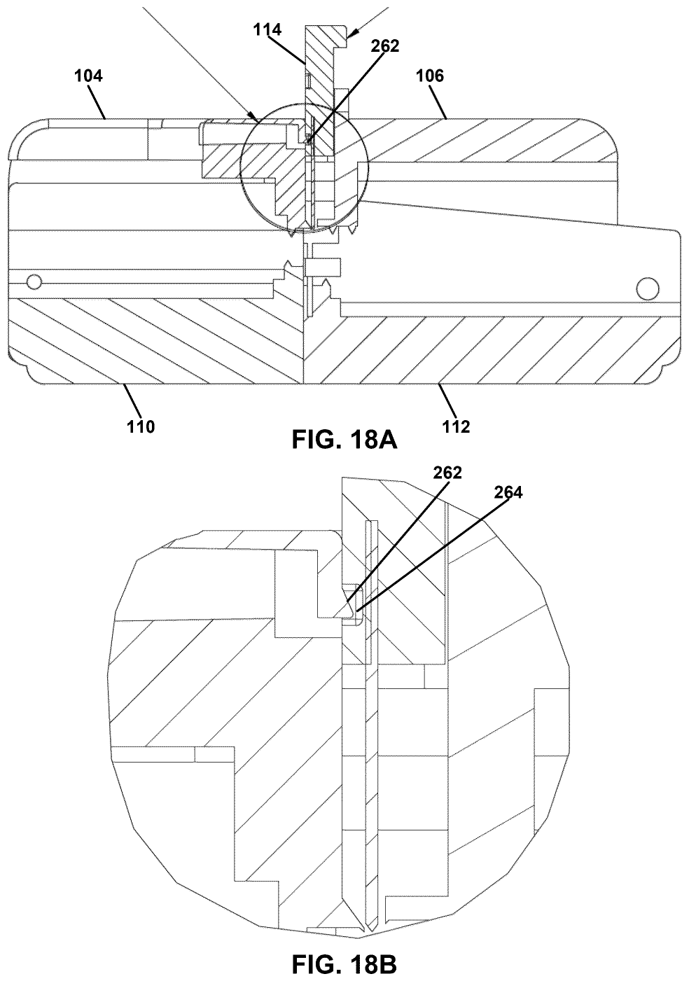

FIGS. 18A-18B depict an anti-rotation mechanism 262 for preventing first top component 104 and bottom component 110 from rotating relative to second top component 106 and second bottom component 112 before cutting instrument 114 advances into disconnector device 100 so that blade 116 cuts flexible tubing 101. As depicted in FIGS. 18A-18B, anti-rotation mechanism 262 can extend into a recess 264 within cutting instrument 114 while cutting instrument 114 is in the retracted position so that anti-rotation mechanism 262 prevents first top component 104 and first bottom component 110 from rotating relative to second top component 106 and second bottom component 112 while cutting instrument 114 is retracted. When cutting instrument 114 is actuated, however, anti-rotation mechanism 262 is no longer within recess 264 and first top component 104 and first bottom component 110 can freely rotate relative to second top component 106 and second bottom component 112.

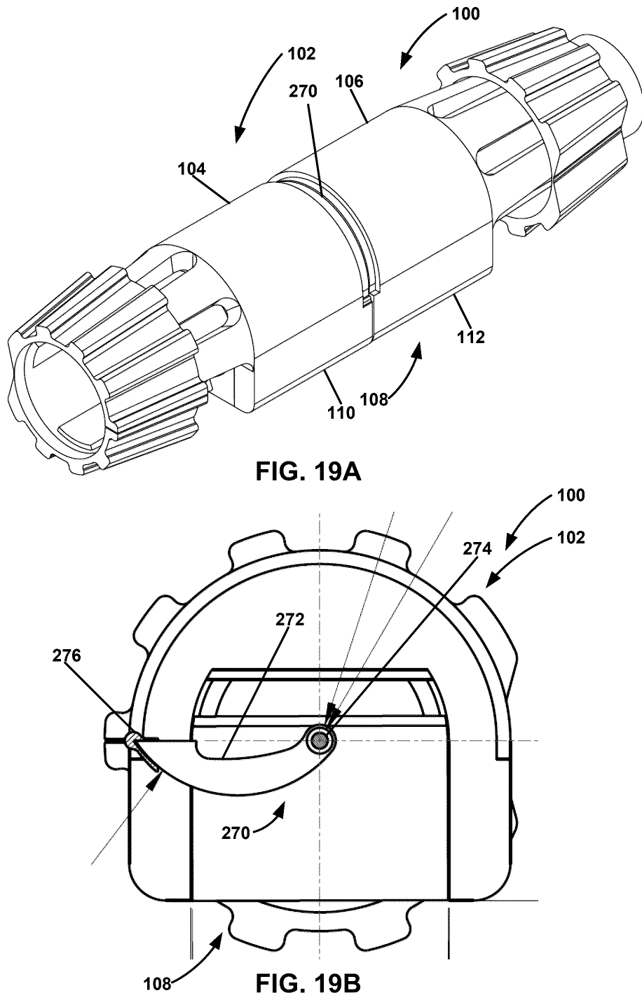

FIGS. 19A-19B depict an embodiment of a disconnector device 100 that includes a cutting instrument 270 that rotates from a central axis. For example, cutting instrument can include a curved blade 272 that rotates about axis 274. The user can, for example, actuate knob 276 to rotate curved blade 272 in a clockwise direction, causing blade 272 to cut through flexible tubing that is compressed by a top structure 102 and a bottom structure 108 of the disconnector device 100. To actuate knob 276, the user can move a thumb around and outer surface of disconnector device 100 so that knob 276 traverses an outer circumference of top structure 102 from the position depicted in FIG. 19B to a position 180 degrees removed from the position of FIG. 19B.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the claim scope. Also, while the systems and methods described herein have been described in reference to some exemplary embodiments, these embodiments are not limiting and are not necessarily exclusive of each other, and it is contemplated that particular features of various embodiments may be omitted or combined for use with features of other embodiments while remaining within the claim scope.

1. A device for cutting a tube, the device comprising:
an outer housing comprising:
a bottom structure; and
a top structure attachable to the bottom structure at a first interface region, the bottom structure and the top structure defining a lumen sized to receive a tube; and
a cutting instrument slidably mounted within an aperture of the top structure to move from a retracted position to an actuated position to cut the tube, wherein a blade of the cutting instrument remains fully within the housing both when the cutting instrument is in the retracted position and when the cutting instrument is in the actuated position,
wherein the outer housing is separable at a second interface region spaced apart from the first interface region when the bottom structure is attached to the top structure.

2. The device of claim 1, wherein the bottom structure is configured to occupy an open rotational position relative to the top structure and a closed rotational position relative to the top structure, and wherein based on the bottom structure transitioning from the open rotational position to the closed rotational position, the bottom structure rotates relative to the top structure about a hinge.

3. The device of claim 2,
wherein the bottom structure defines one or more hinge post receptacles corresponding to the hinge, and
wherein the top structure defines one or more hinge posts for engaging with the one or more hinge post receptacles so that the bottom structure is rotatable relative to the top structure about the hinge.

4. The device of claim 1,
wherein the bottom structure defines one or more tabs that protrude outward from an outer wall of the bottom structure, and
wherein the top structure defines one or more grooves that depress into an inner wall of the top structure such that the one or more tabs engage with the one or more grooves to attach the bottom structure to the top structure at the first interface region.

5. The device of claim 1, wherein the bottom structure is inseparably attachable to the top structure.

6. The device of claim 1,
wherein the bottom structure comprises a first set of pinching teeth, and
wherein the top structure comprises a second set of pinching teeth facing the first set of pinching teeth so that when the bottom structure is attached to the top structure over the tube, the first set of pinching teeth and the second set of pinching teeth compress the tube at a compression zone.

7. The device of claim 6, wherein the first set of pinching teeth and the second set of pinching teeth compress the tube at the compression zone so that liquid cannot flow through the tube across the compression zone.

8. The device of claim 6, wherein the first set of pinching teeth comprise two pinching teeth extending perpendicular to the tube adjacent the second interface, and wherein the second set of pinching teeth comprise two pinching teeth extending perpendicular to the tube adjacent the second interface.

9. The device of claim 1, wherein the cutting instrument comprises the blade and a handle fixedly attached to the blade, wherein a first portion of the handle extends outward from the outer housing when the cutting instrument is in the retracted position, wherein a second portion of the handle extends outward form the outer housing when the cutting instrument is in the actuated position, the second portion being smaller than the first portion.

10. The device of claim 1, wherein the bottom structure and the top structure compress the tube at a compression region when the bottom structure is attached to the top structure at the first interface region, and wherein the second interface region is perpendicular to the tube within the compression region.

11. The device of claim 10, wherein based on the cutting instrument sliding from the retracted position to the actuated position, the blade cuts through the tube within the compression region to separate the tube into a first tube section on a first side of the cut and a second tube second on a second side of the cut.

12. The device of claim 11, wherein the bottom structure and the top structure maintain compression on the first tube section and the second tube section after the cut so that liquid cannot flow from the first tube section and the second tube section through the cut.

13. The device of claim 1, wherein the outer housing comprises:
a third housing portion including a left section of the bottom structure and a left section of the of the top structure; and
a fourth housing portion including a right section of the bottom structure and a right section of the top structure,
wherein the outer housing is separable at the second interface region between the third housing portion and the fourth housing portion.

14. The device of claim 13, wherein the cutting instrument is attached to the fourth housing portion so that when the third housing portion detaches from the fourth housing portion, the cutting instrument remains attached to the fourth housing portion.

15. The device of claim 13, wherein the third housing portion defines a set of receptacles, wherein the fourth housing portion defines a set of posts for engaging with the set of receptacles so that the third housing portion is removably attached to the top structure.

16. The device of claim 15, wherein the set of receptacles comprises two receptacles on the left section of the bottom structure and two receptacles on the left section of the top structure, and wherein the set of posts comprises two posts on the right section of the bottom structure and two posts on the right section of the top structure.

17. The device of claim 15, wherein when the outer housing separates at the second interface region between the third housing portion and the fourth housing portion, the fourth housing portion rotates relative to the third housing portion to disengage the set of grooves from the set of receptacles.

18. The device of claim 1, wherein a sharp edge of the blade of the cutting instrument is encased within a pocket of the outer housing when the cutting instrument is in the actuated position.

19. A device for cutting a tube, the device comprising:
an outer housing comprising:
a bottom structure comprising first pinching teeth; and
a top structure comprising second pinching teeth, the top structure attachable to the bottom structure at a first interface region, wherein the bottom structure and the top structure define a lumen sized to receive a tube, and wherein the first pinching teeth and the second pinching teeth compress the tube within the lumen; and
a cutting instrument to cut the compressed tube within a gap defined by the first pinching teeth and the second pinching teeth,
wherein the outer housing is separable at a second interface region spaced apart from the first interface region, the second interface region extending through the gap defined by the first pinching teeth and the second pinching teeth.

20. A device for cutting a tube, the device comprising:
an outer housing comprising:
a bottom structure; and
a top structure movably attachable to the bottom structure at a first interface region, the bottom structure and the top structure defining a lumen sized to receive a tube; and
a cutting instrument to cut the tube within the lumen,
wherein the outer housing is separable at a second interface region spaced apart from the first interface region based on a first side of the outer housing rotating relative to a second side of the outer housing so that connectors of the first side and the second side disengage, the first side of the outer housing remaining attached to the tube on a first end of the cut and the second side of the outer housing remaining attached to the tube on a second end of the cut.

21. A method, comprising:
engaging toolless aseptic disconnector with an exterior of flexible tubing at a user-selected location such that the toolless aseptic disconnector cuts the flexible tubing;
separating the toolless aseptic disconnector into a first part that is retained at a first cut end of the flexible tubing and a second part that is retained at a second cut end of the flexible tubing.

22. A method, comprising:
mounting an aseptic disconnector to an exterior of flexible tubing;
twisting the aseptic disconnector into separate the aseptic disconnector into first and second pieces while the first and second pieces of the aseptic disconnector remain engaged with the flexible tubing.
